SEQUENCE LISTING (2 sequences)

SEQ ID NO: 1 - DNA, 1629 bp, Homo sapiens
atggccctcccttatcatacttttctctttactgttctcttaccccctttcgctctcactgcaccccctccatgctgctgtacaaccagtagctccccttaccaagagtttctatggagaacgcggcttcctggaaatattgatgccccatcatataggagtttatctaagggaaactccaccttcactgcccacacccatatgccccgcaactgctataactctgccactctttgcatgcatgcaaatactcattattggacagggaaaatgattaatcctagttgtcctggaggacttggagccactgtctgttggacttacttcacccataccagtatgtctgatgggggtggaattcaaggtcaggcaagagaaaaacaagtaaaggaagcaatctcccaactgacccggggacatagcacccctagcccctacaaaggactagttctctcaaaactacatgaaaccctccgtacccatactcgcctggtgagcctatttaataccaccctcactcggctccatgaggtctcagcccaaaaccctactaactgttggatgtgcctccccctgcacttcaggccatacatttcaatccctgttcctgaacaatggaacaacttcagcacagaaataaacaccacttccgttttagtaggacctcttgtttccaatctggaaataacccatacctcaaacctcacctgtgtaaaatttagcaatactatagacacaaccagctcccaatgcatcaggtgggtaacacctcccacacgaatagtctgcctaccctcaggaatattttttgtctgtggtacctcagcctatcattgtttgaatggctcttcagaatctatgtgcttcctctcattcttagtgccccctatgaccatctacactgaacaagatttatacaatcatgtcgtacctaagccccacaacaaaagagtacccattcttccttttgttatcagagcaggagtgctaggcagactaggtactggcattggcagtatcacaacctctactcagttctactacaaactatctcaagaaataaatggtgacatggaacaggtcactgactccctggtcaccttgcaagatcaacttaactccctagcagcagtagtccttcaaaatcgaagagctttagacttgctaaccgccaaaagagggggaacctgtttatttttaggagaagaacgctgttattatgttaatcaatccagaattgtcactgagaaagttaaagaaattcgagatcgaatacaatgtagagcagaggagcttcaaaacaccgaacgctggggcctcctcagccaatggatgccctggactctccccttcttaggacctctagcagctataatatttttactcctctttggaccctgtatcttcaacttccttgttaagtttgtctcttccagaattgaagctgtaaagctacaaatagttcttcaaatggaaccccagatgcagtccatgactaaaatctaccgtggacccctggaccggcctgctagactatgctctgatgttaatgacattgaagtcacccctcccgaggaaatctcaactgcacaacccctactacactccaattcagtaggaagcagttag

SEQ ID NO: 2 - protein, 542 aa, Homo sapiens
MALPYHTFLFTVLLPPFALTAPPPCCCTTSSSPYQEFLWRTRLPGNIDAPSYRSLSKGNSTFTAHTHMPRNCYNSATLCMHANTHYWTGKMINPSCPGGLGATVCWTYFTHTSMSDGGGIQGQAREKQVKEAISQLTRGHSTPSPYKGLVLSKLHETLRTHTRLVSLFNTTLTRLHEVSAQNPTNCWMCLPLHFRPYISIPVPEQWNNFSTEINTTSVLVGPLVSNLEITHTSNLTCVKFSNTIDTTSSQCIRWVTPPTRIVCLPSGIFFVCGTSAYHCLNGSSESMCFLSFLVPPMTIYTEQDLYNHVVPKPHNKRVPILPFVIRAGVLGRLGTGIGSITTSTQFYYKLSQEINGDMEQVTDSLVTLQDQLNSLAAVVLQNRRALDLLTAKRGGTCLFLGEERCYYVNQSRIVTEKVKEIRDRIQCRAEELQNTERWGLLSQWMPWTLPFLGPLAAIIFLLLFGPCIFNFLVKFVSSRIEAVKLQIVLQMEPQMQSMTKIYRGPLDRPARLCSDVNDIEVTPPEEISTAQPLLHSNSVGSS

(19) United States
(12) Patent Application Publication
(10) Pub. No.: US 2006/0088820 A1
(43) Pub. Date: Apr. 27, 2006

(54) Detecting superantigen activity in a biological sample

(75) Inventors: Herve Perron, Paris (FR); Monique Lafon, Paris (FR)

Correspondence Address: OLIFF & BERRIDGE, PLC, P.O. BOX 19928, ALEXANDRIA, VA, 22320, US

(73) Assignee: BIOMERIEUX, Marcy L'Etoile (FR)

(21) Appl. No.: 11/261,673

(22) Filed: Oct. 31, 2005

Related U.S. Application Data
(63) Continuation of application No. 09/936,835, filed on Nov. 20, 2001.

(30) Foreign Application Priority Data
Mar. 19, 1999 (FR) 99 03622
Oct. 28, 1999 (FR) 99 13755

(51) Int. Cl. C12Q 1/70; C12Q 1/68

(52) U.S. Cl. 435/5; 435/6

(57) ABSTRACT

Multiple sclerosis or a condition associated with multiple sclerosis is detected by detecting an amount of lymphocytes bearing a Vβ16, Vβ17 or Vβ7 determinant and calculating an amount of expansion or loss of these lymphocytes.

This is a Continuation of application Ser. No. 09/936,835, filed Nov. 20, 2001, which in turn is a National Stage Application of PCT/FR00/00691, filed Mar. 20, 2000. The entire disclosure of the prior application is hereby incorporated by reference herein in its entirety.

Multiple sclerosis (MS) is a chronic disease of the central nervous system in humans, which evolves through a succession of remission phases and exacerbation phases or according to a steady progression, and the anatomopathological characteristic of which consists of the formation of clearly delimited regions of demyelination in the white matter of the brain and spinal cord.

At the histological level, these regions exhibit, at the early stage of the lesional process, degradation of the periaxonal myelin, associated with an effect on the glial cells responsible for this demyelination. Inflammatory macrophage activation involving microglial cells (resident tissue macrophages of the central nervous system), and also probably macrophages originating from infiltrated blood monocytes, is associated with this demyelination process and contributes to the destruction of the myelinated sheets. At the center of the demyelinated region, there is a relative depletion of glial cells, whereas a proliferation of astrocytes develops at the periphery and may invade the demyelinated plaque, generating a fibrous or gliotic plaque. These sclerotic structures are the basis of the name given to the disease.

Another characteristic of these plaques is their virtually systematic association with a vascular element around which they develop.

At the histological level, frequent modification of the blood-brain barrier (BBB) consisting of the capillary endothelium is observed. One of the determining elements in maintaining the BBB consists of the subjacent presence of cytoplasmic extensions of the astrocytes, termed astrocyte end-feet. The astrocyte end-feet probably induce the formation, or allow the maintenance, of tight junction structures which ensure the cohesion of the capillary endothelial barrier forming the BBB. Now, various pathological models make reference to BBB modification and a depletion of astrocyte end-feet.

Moreover, in the lesional process of MS, modification of the BBB contributes to amplifying the associated inflammatory response, by influx of lymphoid cells from the blood circulation. The contribution of the inflammation associated with immune cells is considerable in MS and contributes to the lesional process.

The etiology of MS is the source of current debate because the disease may have multiple causes. Arguments have been put forward in favor of a bacterial, viral or autoimmune hypothesis.

Retroviruses are potentially interesting candidates for the etiological study of multiple sclerosis, by analogy with an ovine disease which is very close to MS and which is induced in sheep by an exogenous retrovirus: the MAEDI VISNA virus. The experimental infection of sheep by intraventricular inoculation of neurovirulent strains of the VISNA virus has made it possible to establish that this virus is responsible for the genesis of the demyelinating infection in sheep.

Many studies have been carried out to support the hypothesis of a viral etiology for the disease, and the discovery that HTLV-1, an oncovirus, is associated with a chronic and progressive myelopathy, tropical spastic paraparesis (TSP), has relaunched the interest in viruses, although it has not been possible to establish a causal link between viruses and multiple sclerosis.

Recently, the studies by H. Perron et al. (Res. Virol. 1989; 140, 551-561; “Current Concepts in Multiple Sclerosis” Wiethöler et al., eds. Amsterdam, Elsevier, 1991, 111-116 and the Lancet 1991; 337, 862-863) have made it possible, using a lumbar puncture of cerebrospinal fluid from an MS patient, to isolate a nonimmortalized line of nonlymphoid cells and to demonstrate the presence of a virus, which has the characteristics of a retrovirus and shows, in particular, a peak corresponding to reverse transcriptase activity in the culture supernatant of this line. Even more recently, these same authors have obtained, from this peak of reverse transcriptase activity, a complementary DNA which corresponds to the pol gene encoding the RT (reverse transcriptase) enzyme. This retrovirus, termed MSRV by the authors, has in particular been characterized at the genomic level in PCT patent application WO 99/02666. The phylogenic analysis, by comparing the sequence of the pol region of MSRV with other pol sequences available in the databases, has made it possible to show that MSRV is close to the ERV-9 (endogenous retrovirus-9) family.

Moreover, F. Beseme et al., in PCT patent application WO 99/02696, have screened a cDNA library using a Ppol-MSRV probe and detected overlapping clones which have enabled them to reconstruct a putative genomic RNA of 7582 nucleotides. This genomic RNA has an R-U5-gag-pol-env-U3-R structure characteristic of retroviruses, and an investigation of several databases has made it possible to show that there is a considerable amount of related genomic sequences (DNA) in the human genome, which are found on several chromosomes. The authors have thus demonstrated the existence of partial structures of the retroviral type in the human genome and envisaged their potential role in autoimmune diseases such as multiple sclerosis. This novel family of endogenous retroviruses has been named HERV-W, because of its structural characteristics. The phylogenic analysis in the pol region has shown that the HERV-W family is phylogenically close to the ERV-9 and RTVL-H families and therefore belongs to the type I endogenous retrovirus family. Moreover, the phylogenic analysis of the open reading frame of env shows that it is closer to type D simian retroviruses and to avian reticuloendotheliosis retroviruses than to type C mammalian retroviruses, suggesting a chimeric C/D genomic structure. The analyses of the phylogenic trees show that the ERV-9 and HERV-W families derive from two independent waves of insertion.

The envelope protein of MSRV-1 encoded by the MSRV-1 env gene and variants thereof has very strong homology with the env protein of HERV-W encoded by the RNA expressed in human placenta, as described in patent application WO-99/02696 in the name of the applicant.

The MSRV retrovirus is genetically related to the HERV-W family.

The MSRV-1 variants have an env gene which advantageously shows at least 90%, preferably at least 95%, or even 98%, homology with that of MSRV-1.

All these elements plead in favor of retroviral elements being involved in multiple sclerosis.

Moreover, it now appears probable that autoimmune manifestations may be induced by the expression of superantigens (SAgs).

Superantigens are molecules capable of binding to class II major histocompatibility complex (MHC) molecules and to peptide sequences characteristic of certain T-cell receptor families (Vβ). These superantigens activate a large number of T clones, independently of the antigenic peptide recognized by their TCR (T-cell receptor) in association with the MHC of the antigen-presenting cell. The consequence of this activation is a polyclonal proliferation or an induction of anergy, or even of apoptosis, in the T-lymphocyte population carrying this Vβ. Superantigens are expression products from microorganisms, such as bacteria and exogenous or endogenous retroviruses.

Some molecules having properties close to certain effects which superantigens are known to have may also be responsible for major immunopathological processes and are termed superantigen-like. In the remainder of the present description, they are included in the term “superantigen”.

The T receptor (TCR) present at the surface of lymphocytes and involved in the recognition of an antigen or of a superantigen consists of two chains, a 40 to 50 kDa α chain and a 35 to 47 kDa β chain, linked to one another via a disulfide bridge. Each polypeptide chain comprises two domains, each of approximately 110 amino acids, which are organized like the domains of the immunoglobulins. They are anchored in the plasma membrane via a transmembrane peptide and have a short cytoplasmic tail. The difference in molecular weight between the two α and β chains is due to the presence of a carbohydrate-containing chain in the N-terminal position of the a chain. The amino acid sequence variability lies in the N-terminal domain of each α and β polypeptide, which are homologs of the variable domains of immunoglobulins. Each domain is encoded by a rearrangement of V, D and J genes for the β chain and V and J genes for the a chain. Analysis of the sequences of these various TCR V domains reveals a great variability which corresponds to the hypervariable regions of immunoglobulins (CDRs). (Roitt I. et al., Immunology 3^{rd} edition, Mosby, England).

The rearrangement of the TCR genes is similar to that of the immunoglobulin genes. The diversity of TCRs originates from genetic recombination between the V, D and J segments. The Vβ genes, including the D, J and C genes, are grouped together, with the exception of Vβ14 which is present at the 3′ end of the locus. Extensive diversity is generated during the processes for grouping together the V-D-J regions, but also the V-J and V-D-D-J regions.

A superantigen is capable of activating T lymphocytes nonspecifically, unlike an antigen. A superantigen is capable of binding jointly to the MHCII molecules present at the surface of antigen-presenting cells and to the Vβ molecules of the T receptor present at the surface of T cells. The Vβ chain of the TCR binds the superantigen outside the antigen-specific site of the TCR, but this is sufficient to activate the T cell. This binding leads to intracellular signal induction in the T cell. These intracellular signal cascades induce either the proliferation of the T cell bearing the Vβ of interest, or anergy or apoptosis of the cell. Thus, depending on the experimental conditions, the effect on the activated T-lymphocyte subpopulation may be an effect of polyclonal apoptosis, anergy or proliferation. Unlike a T response to a conventional antigen, the stimulation of the T lymphocytes is therefore polyclonal and not oligoclonal, or even monoclonal (Scherer et al., 1993 Annu. Rev. Cell Biology 9: 101-128). This induction of cell proliferation, of anergy or of apoptosis depends on several parameters which act in combination. It depends in particular on the concentration of the superantigen and on the existence of prior encounters of analogous stimulations targeting the same Vβ, by the immune system of the T-lymphocyte donor. The induction into a state of anergy or of apoptosis may follow, inter alia, sustained stimulation or activation of the T cells. Thus, a superantigen has an effect which induces a positive or negative variation in a subpopulation of T cells with a defined Vβ “x”. A superantigen which targets a given Vβ “x” antigenic determinant will interact with the entire lymphocyte subpopulation bearing this Vβ. The effect induced by this interaction coupled with that targeting the MHCII of the antigen-presenting cells (APCs) is a significant variation of the percentage of Vβ “x” lymphocytes compared to the other Vβ non-“x” T lymphocytes. This variation is typically either a proliferation or a depletion (Bernal A et al., 1999, J Clin Immunol 19: 149; Li H et al., 1999 Annu Rev Immunol 17: 435; Girgis et al., 1999 J Exp Med 189: 265; Lavoie et al., Immunol Rev 1999 168: 257; Cornwell W D and Rigers T J, Immunology 1999 96: 193; Wang Z Q et al., Immunology 1998 94: 331; Maier C C et al., PNAS 1998 95: 4499; Michie C A and Cohen J, Trends Microbiol 1998 6: 61; La Bon A et al., Int Immunol 1999 11: 373; Shen X and Konig R, Int Immunol 1998 10: 247; Noble A et al., J Immunol 1998 160: 559; Yang Y et al., Int Immunol 1998 10: 175; Renno T et al., J Immunol 1999 162: 6312; Roitt I et al., Immunology third edition, Mosby).

Moreover, when the agent encoding a given superantigen, is placed together with T lymphocytes, and not only together with the purified molecule bearing the superantigen activity, the resulting immunological effect corresponds to a superantigen effect superimposed on varied antigen stimulations caused by the other antigens of the whole agent. It should be noted that, unlike the superantigen stimulation, the latter may differ as a function of the class II HLA of the lymphocyte donor or, in a pathological context, of the patient. As a consequence of this, the Vβ “x” T proliferation or depletion is accompanied by a profile of reactivity (proliferation or inhibition) of other Vβ non-“x” subpopulations, possibly variable according to the HLA II; this profile being defined by the nature of the antigens associated with the agent or with several agents (in the case of a cascade leading to the activation of an endogenous retrovirus encoding the superantigen expressed under these conditions). In the latter case, two superantigens may be expressed if the inducing agent produces one of them and if the infection of a target cell by this agent reactivates an endogenous retrovirus which then produces a second one. These data therefore confirm the necessity of evaluating a complete T response profile as a function of Vβ, when a “natural” context of infection/reactivation is studied rather than a purified superantigen molecule out of context.

The applicant has now shown that the expression of a superantigen, or of a protein having certain effects of the superantigen type (superantigen-like), is associated with a pathological condition, for example a condition associated with multiple sclerosis.

The superantigen activity is induced directly or indirectly by an effector agent, such as a protein or a microorganism or a pathogenic agent, particularly a retrovirus (MSRV-1) and/or a pathogenic agent (MSRV-2), and in particular an epitope included in an env protein of an MSRV-1 retrovirus.

In addition, the applicant has also demonstrated a stimulated production of cytokines, such as IL-6, in mononucleated blood cells which originate from healthy donors and which are brought into contact with an extract, or a fraction of an extract, of MS cell culture supernatant.

The applicant has also demonstrated a profile of T response, as a function of Vβs, to one or more agent(s) associated with the expression of a superantigen-like molecule.

Finally, the applicant has observed that the same extracts originating from patients suffering from MS and/or infected with the MSRV-1 retrovirus induce significantly high death by apoptosis in the same mononucleated blood cells.

The applicant has therefore developed a method for demonstrating the abovementioned effects in a biological sample.

The criteria required to establish that a protein or that a microorganisms is or contains a superantigen or a protein having certain superantigen-like effects are:

(i) the capacity of the protein or of the microorganism to induce the expansion or the loss of certain lymphocyte families bearing, within their TCR, a particular Vβ chain, and

(ii) Vβ activation independent of the class II MHC haplotype.

It should be noted that, when reference is made to superantigen activity in the present invention, this means equally the expression of a superantigen or of a protein having certain superantigen-like effects (SAg-like).

The invention relates to a method for detecting superantigen activity in a biological sample, according to which a majority expansion of lymphocytes bearing a Vβ16 and/or Vβ17, preferably Vβ16, determinant is demonstrated.

According to an advantageous variant, a profile of a majority expansion of lymphocytes bearing a Vβ16 determinant and of a co-expansion of lymphocytes bearing Vβs chosen from at least any one of Vβ2, Vβ3, Vβ7, Vβ8, Vβ12, Vβ14, Vβ7 and Vβ22, and preferably of Vβ3 and Vβ12, is demonstrated.

The invention also relates to a method for detecting superantigen activity, in which a majority loss of lymphocytes bearing a Vβ16 and/or Vβ17 determinant is demonstrated.

According to an advantageous variant, a majority loss of lymphocytes bearing a Vβ16 determinant and a co-decrease of lymphocytes bearing Vβs chosen from at least any one of Vβ2, Vβ3, Vβ7, Vβ8, Vβ12, Vβ14, Vβ17 and Vβ22, preferably of Vβ7, Vβ4 and Vβ17, and advantageously of Vβ7 and Vβ17, are demonstrated.

The expansion and, optionally, the co-expansion, or the loss and, optionally, the co-decrease, is detected by revealing the membrane-bound Vβ molecules associated with the blood mononucleated cells, which are preferably T lymphocytes. A percentage of variation of the Vβ molecules detected and originating from all or part of the culture supernatant from patients suffering from MS is then calculated relative to the number of Vβ molecules detected in all or part of a culture supernatant originating from a healthy donor. A mixture of culture supernatants originating from healthy donors can also be envisaged.

The set of Vβ molecules is termed the “Vβ repertoire” of the T lymphocytes. The various Vβ molecules are detected specifically, using at least one of the techniques described below:

(a) either using at least one ligand, i.e. any molecule capable of recognizing specifically the Vβ to be detected, for example an anti-Vβ monoclonal or polyclonal antibody, or a monoclonal or polyclonal antibody fragment, or a molecule which inhibits the function of the Vβ under consideration. The percentage of Vβ molecules of the repertoire is then related to the percentage of cells exhibiting the CD3 molecule at their surface, the latter also being detected specifically using at least one ligand. The term “ligand” is intended to mean in particular a monoclonal or polyclonal antibody, or a fragment of said antibodies, preferably a monoclonal antibody. The monoclonal antibodies directed against a Vβ of interest are produced by conventional techniques used to produce antibodies against surface antigens. Mice or rabbits are immunized (i) either with a natural or recombinant protein, (ii) with an immunogenic peptide, (iii) or with murine cells which express the protein or the peptide of interest and MHCII molecules. The Balb/c murine line is the most commonly used. The immunogen may also be a peptide chosen from the peptides defined from the primary sequences of the Vβs of interest. The proteins or peptides are coupled to keyhole limpet hemocyanin (peptide-KLH), as a support for their use in immunization, or coupled to human serum albumin (peptide-HSA). The animals are given an injection of peptide-KLH or of peptide-HSA, using complete Freund's adjuvant (IFA). The sera and the hybridoma culture supernatants derived from the animals immunized with each peptide are analyzed for the presence of antibodies with an ELISA assay using the initial molecules. The spleen cells of these mice are recovered and fused with myeloma cells. Polyethylene glycol (PEG) is the most commonly used fusion agent. The hybridomas producing the most specific and the most sensitive antibodies are selected. The monoclonal antibodies can be produced in vitro by cell culture of the hybridomas produced or by recovering murine ascites fluid after intraperitoneal injection of the hybridomas into mice. Whatever the method of production as supernatant or as ascites, it is then important to purify the monoclonal antibody. The purification methods used are essentially filtration over ion exchange gel or by exclusion chromatography, or even immunoprecipitation. For each antibody, the method which will make it possible to obtain the best yield should be chosen. A sufficient number of antibodies is screened in functional assays in order to identify the antibodies which are the most effective in binding the molecule of interest and/or in blocking the activity of the molecule of interest. The selected monoclonal antibodies are humanized using standard “CDR grafting” methods (protocol carried out by many companies, in the form of a service). These humanized antibodies can be tested clinically in patients. The effectiveness of these antibodies can be monitored using clinical parameters. The in vitro production of antibodies, of antibody fragments or of antibody derivatives, such as chimeric antibodies which may or may not be humanized and which are produced by genetic engineering, in eukaryotic cells has been described (EP 120 694 or EP 125 023) and can also be applied to the present invention;

(b) or by molecular biology, after extracting the nucleic acids from blood mononucleated cells, inter alia T lymphocytes, which have been placed together with MS culture supernatant or a fraction of MS culture supernatant and together with a control culture supernatant or a fraction of control culture supernatant, amplifying their transcribed Vβ RNAs by RT-PCR, studying the profile of the amplification products on a gel and/or sequencing and/or electrophoresis. For the RT-PCR step and/or for specifically detecting the amplification products, use is made of DNA and/or RNA fragments encoding the Vβ determinant studied, or a fragment of this determinant, and/or DNA and/or RNA fragments capable of hybridizing and of amplifying nucleic acid fragments encoding the Vβ determinant studied, by complementarity of the nucleic acid bases. The profile of the amplified fragments, the size of which varies as a function of the rearrangement of the chains specific for each clone, is determined by electrophoretic analysis and makes it possible to objectify a polyclonality. It is also possible to detect the amplified DNA and/or RNA fragments encoding the Vβ determinant studied, by nucleotide hybridization according to the techniques known to those skilled in the art (Southern Blot, Northern Blot, ELOSA “Enzyme-Linked Oligosorbent Assay” (Katz J B et al., Am. J. Vet. Res. 1993 December; 54 (12): 2021-6 and François Mallet et al., Journal of Clinical Microbiology, June 1993, p 1444-1449)).

Thus, a subject of the present invention is an advantageous method for demonstrating the abovementioned superantigen activity, which comprises the following steps:

(i) a culture supernatant of blood mononucleated cells, of choroid plexus cells or of leptomeningeal cells, said cells originating from patients suffering from an autoimmune disease, or of an established cell line, such as the cells of the PLI-2 cell line deposited at the ECACC on Jul. 22, 1992, under the number 92072201 and the LM7PC cell line deposited at the ECACC on Jan. 8, 1993, under the number 93010817, in accordance with the provisions of the Treaty of Budapest, is sampled, and

(ii) said culture supernatant, or a part of the culture supernatant is brought into contact with a series of cultures, preferably at least three, of blood mononucleated cells originating from healthy donors, and

(iii) said expansion and, optionally, a co-expansion, or said loss and, optionally, co-decrease of the blood mononucleated cells of step (ii) are detected.

In particular, the patients suffering from an autoimmune disease or suspected of having a risk of developing the disease are MS patients and the blood mononucleated cells which produce superantigen molecules and which originate from patients suffering from MS are in particular chosen from B lymphocytes and monocytes.

The blood mononucleated cells which respond to the stimulation and which originate from healthy donors are in particular chosen from T lymphocytes.

Another advantageous method of the invention consists in

(i) sampling blood mononucleated cells, said cells originating from patients suffering from an autoimmune disease or from patients suspected of having a risk of developing an autoimmune disease, and from healthy individuals,

(ii) bringing said blood mononucleated cells originating from patients or from healthy individuals into contact with culture supernatants, or a fraction of culture supernatant, of cells chosen from blood mononucleated cells, choroid plexus cells and leptomeningeal cells, and cells derived from established cell lines, such as the cells of the PLI-2 cell line deposited at -the ECACC on Jul. 22, 1992, under the number 92072201 and the LM7PC cell line deposited at the ECACC on Jan. 8, 1993, under the number 93010817, in accordance with the provisions of the Treaty of Budapest, and

(iii) detecting said expansion and, optionally, co-expansion, or said loss and, optionally, co-decrease, using the blood mononucleated cells of step (i).

In particular, the cells originate from patients suffering from multiple sclerosis or suspected of having a risk of developing the disease, or derive from a culture of cells derived from MS patients.

Said expansion and, optionally, co-expansion, or said loss and, optionally, co-decrease, are demonstrated

(a) either using ligands, in particular an antibody, and preferably a monoclonal antibody or an antibody fragment, each ligand being specific for a determinant chosen from Vβ16, Vβ2, Vβ3, Vβ7, Vβ8, Vβ12, Vβ14, Vβ17 and Vβ22, preferably Vβ16, Vβ3 and Vβ12, or a ligand, in particular an antibody, and preferably a monoclonal antibody, each ligand being specific for a determinant chosen from Vβ16, Vβ2, Vβ3, Vβ7, Vβ8, Vβ12, Vβ14, Vβ17 and Vβ22, preferably Vβ16, Vβ7, Vβ14 and Vβ17,

(b) or according to the protocol described below by carrying out:

(i) extraction of the total RNAs from the blood mononucleated cells which have been placed together with MS culture supernatant or a fraction of MS culture supernatant and together with control culture supernatant or a fraction of control culture supernatant,

(ii) reverse transcription of said RNAs,

(iii) amplification specific for each Vβ family using a given pair of primers,

(iv) labeling of the amplification products obtained, with any suitable label,

(v) electrophoresis of said amplification products and analysis of the electrophoretic profiles obtained, using a suitable detector.

The predisposition to a pathological condition is evaluated by experimentally testing the immunological sensitization of a patient by demonstrating a majority expansion or loss of lymphocytes bearing a Vβ16 and/or Vβ17 determinant and, optionally, a co-expression or a co-decrease of lymphocytes bearing Vβs chosen from at least any one of Vβ2, Vβ3, Vβ7, Vβ8, Vβ12, Vβ14, Vβ17 and Vβ22, preferentially of Vβ3 and Vβ12 or of Vβ7, Vβ14 and Vβ17 preferably Vβ7 and Vβ17.

The predisposition detected may be a predisposition which is natural or a predisposition which is acquired, for example after a strong stimulation in an individual of T lymphocytes bearing Vβ16 or Vβ17, preferably Vβ16, determinants, by exposure of the individual's body to an antigen. This antigen may, for example, be at least one protein or one peptide of the hepatitis B virus.

Thus, a subject of the present invention is a method for detecting a pathological condition or a predisposition to a pathological condition, according to which:

a majority expansion or loss of lymphocytes bearing a Vβ16 and/or Vβ17, preferentially Vβ16, determinant, or majority expansion or loss of lymphocytes bearing a Vβ16 determinant and a co-expansion or co-decrease of lymphocytes bearing T-lymphocyte Vβs chosen from at least any one of Vβ2, Vβ3, Vβ7, Vβ8, Vβ12, Vβ14, Vβ17 and Vβ22, and preferentially Vβ3 and Vβ12 or Vβ7, Vβ14 and Vβ17, preferably Vβ7 and Vβ17, are determined, and

a percentage of variation of the number of Vβs detected in a patient suffering from MS is calculated relative to that obtained with the T lymphocytes of a healthy donor, under conditions as determined above.

If the percentage of variation, in absolute value, is significantly different from 0, it may be deduced therefrom that the patient has a predisposition for the disease multiple sclerosis and/or is developing the disease. If this percentage, in absolute value, is equal to 0, this may mean that the donor has no predisposition for the disease multiple sclerosis and/or has not developed the disease at the time at which the method was used.

A subject of the invention is also a method for detecting a pathological condition or a predisposition to a pathological condition, in a biological sample, according to which at least one of the following parameters is demonstrated:

superantigen activity, as defined above,

stimulation of the production of cytokines, such as IL-6,

induction of cellular apoptosis.

Preferably, at least two of the parameters are detected in combination, in particular superantigen activity and induction of apoptosis, or superantigen activity and stimulation of the production of cytokines are detected.

Even more advantageously, the three parameters are detected in combination.

The pathological condition is in particular associated with an autoimmune disease, such as multiple sclerosis.

The superantigen activity detected according to the methods of the invention may in particular be induced directly or indirectly by an effector agent chosen from proteins, microorganisms and pathogenic agents, and for example bacteria and/or retroviruses, preferably human retroviruses, such as MSRV-1, and/or pathogenic agents such as MSRV-2.

In particular, the superantigen activity is induced by the envelope protein of MSRV-1 referenced in SEQ ID No. 2 or by a fragment of said protein, or alternatively by the env gene of MSRV-1 referenced in SEQ ID No. 1 or a fragment of said gene.

The invention also relates to:

a human retrovirus, in particular an endogenous retrovirus, which has superantigen activity and is associated with an autoimmune disease, according to which the retrovirus is MSRV-1 and the superantigen activity is induced by the expression of the env gene of MSRV-1 or of a fragment of said gene, in particular a fragment of said gene encoding at least one reading frame of the env protein of MSRV-1 (SEQ ID No. 2), or alternatively it is induced by the env protein of MSRV-1 or by a fragment of said protein, in particular by a fragment corresponding to at least one reading frame of said protein (SEQ ID No. 2);

a nucleic acid molecule comprising at least one or more fragment(s) of the RNA or of the DNA of the env gene of MSRV-1, identified by SEQ ID No. 1, said fragment being at least 18 nucleotides, and preferably at least 24 nucleotides, in length; in particular it comprises at least one fragment encoding at least one reading frame and optionally containing a stop codon; this molecule possibly encoding superantigen activity;

1a polypeptide molecule, in particular protein or protein fragment comprising at least one or more fragment(s) of said env protein of MSRV-1 identified by SEQ ID No. 2, said fragment being at least 6 amino acids, and preferably at least 8 amino acids, in length; advantageously, the polypeptide molecule comprises at least one reading frame and optionally has superantigen activity;

a vector comprising nucleic acid molecules as defined above.

The invention also relates to a method for detecting superantigen activity in a biological sample from patients suffering from multiple sclerosis, according to which a majority expansion or significant loss of lymphocytes bearing a Vβ7 determinant is demonstrated. To this end, the following procedure may be carried out:

(i) a culture supernatant of blood mononucleated cells, of choroid plexus cells or of leptomeningeal cells, said cells originating from patients suffering from an autoimmune disease, or of an established cell line, such as the cells of the PLI-2 cell line deposited at the ECACC on Jul. 22, 1992, under the number 92072201 and the LM7PC cell line deposited at the ECACC on Jan. 8, 1993, under the number 93010817, in accordance with the provisions of the Treaty of Budapest, is sampled, and

(ii) said culture supernatant, or a part of the culture supernatant, is brought into contact with a series of cultures, preferably at least three, of blood mononucleated cells originating from healthy donors, and

(iii) said expansion and, optionally, a co-expansion, or said loss and, optionally, co-decrease of the blood mononucleated cells of step (ii) are detected, as described above using a ligand or by amplification combined with electrophoresis;

or

(i) blood mononucleated cells are sampled, said cells originating from patients suffering from an autoimmune disease or from patients suspected of having a risk of developing an autoimmune disease, in particular MS, and from healthy individuals,

(ii) said blood mononucleating cells originating from patients or from healthy individuals are brought into contact with culture supernatants, or a fraction of culture supernatant, of cells chosen from blood mononucleated cells, choroid plexus cells and leptomeningeal cells, and cells derived from established cell lines, such as the cells of the PLI-2 cell line deposited at the ECACC on Jul. 22, 1992, under the number 92072201 and the LM7PC cell line deposited at the ECACC on Jan. 8, 1993, under the number 93010817, in accordance with the provisions of the Treaty of Budapest, and

(iii) said expansion and, optionally, co-expansion, or said loss and, optionally, co-decrease, using the blood mononucleated cells of step (i), are detected.

The invention also relates to a method for detecting superantigen activity of the invention, according to which

(i) a polypeptide, in particular a recombinant protein, as identified by SEQ ID No. 2, or a fragment of said polypeptide or of said protein, is produced or synthesized,

(ii) said polypeptide or said protein is brought into contact with a series of cultures, preferably at least three, of blood mononucleated cells originating from healthy donors, and

(iii) said expansion and, optionally, a co-expansion, or said loss and, optionally, co-decrease, of the blood mononucleated cells of (ii) are detected.

Or alternatively according to which

(i) blood mononucleated cells are sampled, said cells originating from patients suffering from an autoimmune disease or from patients suspected of having a risk of developing an autoimmune disease, in particular MS, and from healthy individuals,

(ii) said mononucleated cells originating from patients or from healthy individuals are brought into contact with a polypeptide or a recombinant protein, and

(iii) said expansion and, optionally, co-expansion, or said loss and, optionally, co-decrease, using the blood mononucleated cells of (i) are detected.

Preferably, a polypeptide of the invention as defined above is used. Advantageously, it is encoded by a nucleic acid of the invention as defined above or by a vector of the invention as defined above.

The methods as defined above may be used for the therapeutic monitoring of patients and/or the evaluation of the effectiveness of molecules for therapeutic use, with respect to identified parameters.

In order to evaluate the effectiveness of the molecules for therapeutic use, i.e. one or more molecule(s) capable of inhibiting the expansion or the loss of the T lymphocytes of a given Vβ,

(i) blood mononucleated cells are sampled, said cells originating from patients suffering from an autoimmune disease or suspected or having a risk of developing the disease, in particular MS, and from healthy individuals,

(ii) said blood mononucleated cells originating from MS patients or from healthy individuals are brought into contact with culture supernatants, or a fraction of culture supernatant, of cells chosen from blood mononucleated cells, choroid plexus cells, leptomeningeal cells and cells derived from established cell lines, such as the cells of the PLI-2 cell line and the LM7PC cell line, and

(iii) the inhibition of said expansion and, optionally, co-expansion, or the inhibition of said loss and, optionally, co-decrease, of the lymphocytes bearing at least one determinant chosen from Vβ16, Vβ2, Vβ3, Vβ7, Vβ8, Vβ12, Vβ14, Vβ17 and Vβ22, in particular Vβ16 and/or Vβ17, Vβ16, Vβ3 and Vβ12 or Vβ16, Vβ7, Vβ14 and Vβ17, particularly Vβ16, Vβ7 and Vβ17, using the blood mononucleated cells of step (i), in the presence of said molecule at given doses, are detected using a ligand as described above or amplification combined with electrophoresis as described above.

A percentage of variation of the number of Vβs detected in the presence of the molecule(s) for therapeutic use and in the absence of such molecule(s) are thus calculated.

The molecule(s) for therapeutic use is (are) also tested in vivo according to a method which consists

in using a culture supernatant of mononucleated cells, preferably B lymphocytes and monocytes, of choroid plexus cells or of leptomeningeal cells, said cells being isolated from a biological sample from a patient suffering from MS, in using the supernatant of an established cell line, such as the PLI-2 cell line and the LM7PC cell line, or in using all or part of at least one molecule obtained from at least one of the two abovementioned supernatants,

in bringing all or part of at least one of the abovementioned supernatants or at least one molecule contained in at least one of them into contact with a culture of blood mononucleated cells, preferably T lymphocytes, sampled from an individual and/or an animal before and after administration of the molecule(s) to be tested and, in parallel, after administration of a placebo agent and/or composition to an MS patient and to an animal, in demonstrating, as described above, the expansion or loss of the lymphocytes having the Vβ determinant, before and after administration of the molecule(s) for therapeutic use, or the placebo agent and/or composition, to the individual and/or to the animal,

in detecting said expansion and, optionally, a co-expansion, and/or the loss and, optionally, a co-decrease, of the T lymphocytes expressing at their surface a given Vβ molecule, using at least one ligand or by molecular biology as described above, and

in calculating a percentage (X) of expansion or of loss of Vβ determinants detected in the mononucleated cells placed in culture and derived from the individual or from the animal which has received one or more administrations of the molecule(s) to be tested, relative to the number of Vβ determinants detected in the cells placed in culture and derived from the individual or from the animal which has received no administration, and/or a percentage (Y) of expansion or of loss of Vβ determinants detected in the mononucleated cells placed in culture and derived from the individual or from the animal which has received one or more administrations of the molecule(s) to be tested, relative to the number of Vβ determinants detected in the cells placed in culture and derived from the patient or from the animal which has received one or more administration(s) of molecule(s) and/or of a placebo composition or agent, respecting the same administration conditions as those used for the molecule(s) to be evaluated.

If |X| and/or |Y| and/or |X|+|Y| are significantly different from 0, this may mean that the molecule(s) totally or partially inhibit(s) the expansion or the loss of mononucleated cells with the Vβ determinant under consideration; if |X| or |Y| or |X|+|Y| are equal or close to 0, this may mean that the agent does not totally or only partially inhibits the expansion or the loss of mononucleated cells with the Vβ determinant under consideration.

The term “placebo agent and/or composition” is intended to mean any agent and/or composition which does not cause a decrease in the blood mononucleated cells which have the given Vβ determinant.

The invention also relates to a method for evaluating the effectiveness of an agent or of a composition in inhibiting superantigen activity in a biological sample, comprising the following steps:

(i) a polypeptide, in particular a recombinant protein as defined by SEQ ID No. 2, or a fragment of said polypeptide or of said protein, is produced or synthesized,

(ii) said polypeptide or recombinant protein is brought into contact with a series of cultures, preferably at least three, of blood mononucleated cells originating from healthy donors, in the presence of said agent or of said composition at predetermined doses, and

(iii) the inhibition of said expansion and, optionally, co-expansion, or the inhibition of said loss and, optionally, co-decrease, of the lymphocytes bearing at least one determinant chosen from Vβ16, Vβ2, Vβ3, Vβ7, Vβ8, Vβ12, Vβ14, Vβ17 and Vβ22, in particular Vβ16 and/or Vβ17, Vβ16, Vβ3 and Vβ12 or Vβ16, Vβ7, Vβ14 and Vβ17, particularly Vβ16, Vβ7 and Vβ17, are detected using a ligand or amplification combined with electrophoresis as described above.

A subject of the invention is also a method for evaluating the effectiveness of a therapeutic agent and/or composition in a biological sample, or after administration to a patient or an animal, with respect to a pathological condition. According to this method, an inhibition or a decrease of superantigen activity, as defined above, is demonstrated.

The therapeutic effectiveness of an agent is determined by estimating the inhibition or the decrease of the superantigen activity, which is itself determined by the inhibition of the expansion and/or the decrease or by the inhibition and/or the decrease of the T lymphocytes bearing given Vβs.

To do this:

(i) a culture supernatant, or a fraction of culture supernatant, of blood mononucleated cells, of choroid plexus cells or of leptomeningeal cells, said cells originating from patients suffering from an autoimmune disease, in particular MS, or of cells of an established cell line, such as the cells of the PLI-2 cell line and the LM7PC cell line, is sampled,

(ii) said culture supernatant is brought into contact with a series of cultures of blood mononucleated cells originating from healthy donors, in the presence of said agent or of said composition at predetermined doses, and

(iii) the inhibition of said expansion and, optionally, co-expansion, or the inhibition of said loss and, optionally, co-decrease, of the lymphocytes bearing at least one determinant chosen from Vβ16, Vβ2, Vβ3, Vβ7, Vβ8, Vβ12, Vβ14, Vβ17 and Vβ22, in particular Vβ16 and/or Vβ17, Vβ16, Vβ3 and Vβ12 or Vβ16, Vβ7, Vβ14 and Vβ17, particularly Vβ16, Vβ7 and Vβ17, are detected using a ligand or amplification combined with electrophoresis as described above, or

(i) blood mononucleated cells are sampled, said cells originating from patients suffering from an autoimmune disease, in particular MS patients, and from healthy individuals,

(ii) said blood mononucleated cells originating from patients and from healthy individuals are brought into contact with culture supernatants, or a fraction of culture supernatant, of cells chosen from blood mononucleated cells, choroid plexus cells, leptomeningeal cells and cells derived from established cell lines, such as the cells of the PLI-2 cell line and the LM7PC cell line, and

(iii) the inhibition of said expansion and, optionally, co-expansion, or the inhibition of said loss and, optionally, co-decrease, of the lymphocytes bearing at least one determinant chosen from Vβ16, Vβ2, Vβ3, Vβ7, Vβ8, Vβ12, Vβ14, Vβ117 and Vβ22, in particular Vβ16 and/or Vβ17, Vβ16, Vβ3 and Vβ12 or Vβ16, Vβ7, Vβ14 and Vβ17, particularly Vβ16, Vβ7 and Vβ17, using the blood mononucleated cells of step (i), in the presence of said agent or of said composition at given doses, are detected using a ligand as described above or amplification combined with electrophoresis as described above.

The therapeutic effectiveness of several agents and/or compositions may be evaluated in combination in the same assay.

An advantageous in vivo method for testing the effectiveness of a therapeutic agent in multiple sclerosis comprises the following steps:

the percentages X and Y as described and defined above are determined. X and Y are determined after administration of an agent and/or composition to be evaluated to a patient suffering from MS,

similarly, X′ and Y′ are determined; they are determined in the same way as X and Y, respectively, but after administration of the same compound to a healthy individual,

the percentages x and y, with x=X/X′ and y=Y/Y′, are calculated. These percentages reflect the decrease in the number of blood mononucleated cells exhibiting the Vβ determinant, due to the administration of the therapeutic compound to be tested to a patient suffering from multiple sclerosis, relative to a healthy individual.

If |x| and/or |y| and/or |x|+|y| are significantly different from 0, this may mean that the agent and/or the composition tested at a therapeutic effect with respect to the MS pathological condition; if |x| and/or |y| and |x|+|y| are equal or close to 0, this may mean that the agent and/or the composition tested has very little or no therapeutic effect with respect to the MS pathological condition.

Another method for evaluating the effectiveness of an agent or of a composition in inhibiting superantigen activity in a biological sample comprises the following steps:

(i) blood mononucleated cells are sampled, said cells originating from patients suffering from an autoimmune disease or suspected of having a risk of developing the disease, in particular MS, and from healthy individuals,

(ii) said blood mononucleated cells originating from patients or from healthy individuals are brought into contact with a polypeptide or a recombinant protein, preferably as identified by SEQ ID No. 2, or a fragment of said polypeptide or of said protein, and

(iii) the inhibition of said expansion and, optionally, co-expansion, or the inhibition of said loss and, optionally, co-decrease, of the lymphocytes bearing at least one determinant chosen from Vβ16, Vβ2, Vβ3, Vβ7, Vβ8, Vβ12, Vβ14, Vβ17 and Vβ22, in particular Vβ16 and/or Vβ17, Vβ16, Vβ3 and Vβ12 or Vβ16, Vβ7, Vβ14 and Vβ17, particularly Vβ16, Vβ7 and Vβ17, using the blood mononucleated cells of step (i), in the presence of said agent or of said composition at given doses, are detected using a ligand or amplification combined with electrophoresis as described above.

Preferably, the cells originate from a patient suffering from an autoimmune disease, in particular multiple sclerosis, and/or the blood mononucleated cells which originate from patients suffering from MS are chosen from B lymphocytes and monocytes.

A subject of the invention is also a composition for therapeutic and/or prophylactic use, which comprises, inter alia, a therapeutic agent capable of inhibiting, directly or indirectly, superantigen activity in a biological sample, as defined above, optionally in combination with a vehicle and/or excipient and/or adjuvant and/or diluent which is (are) pharmaceutically acceptable, i.e. which allow(s) administration to animals and to humans. All these data form part of the general knowledge of those skilled in the art (see, for example, Remington's Pharmaceutical Sciences 16^{th} ed. 1980, Mack Publishing Co). The pharmaceutically acceptable vehicle is preferentially isotonic, hypotonic or exhibits weak hypertonicity and has a relatively low ionic strength, such as for example a sucrose solution. Moreover, said composition may contain solvents, aqueous or partially aqueous vehicles, such as sterile water free of pyrogenic agents, and dispersion media, for example. The pH of these pharmaceutical compositions is suitably adjusted and buffered according to conventional techniques.

The term “therapeutic effectiveness” is intended to mean the clinical and biological benefit acquired after administration of a therapeutic agent for the purpose of improving, or even curing the disease. This benefit results, inter alia, in an attenuation of the clinical and biological symptoms and of the pathological effects of the disease after a clinical analysis by the physician and/or biological analyses, such as magnetic resonance imaging, analysis of the oligoclonal bands in the cerebrospinal fluid, analysis of potentials, etc. This attenuation of clinical symptoms and pathological effects should lead to a benefit for the patient (Schwartz and Lazar, 1995, Elements de statistique médicale et biologique [Elements of Medical and Biological Statistics], eds Flammarion; Lazar and Schwartz, 1995, Elements de statistique médicale et biologique [Elements of Medical and Biological Statistics], eds Flammarion). The disease preferably studied is multiple sclerosis. These therapeutic agents are capable (i) of qualitatively and/or quantitatively influencing the superantigen activity as described in the present invention and/or (ii) of modulating and/or of inhibiting the expression of Vβ determinants identified in the present invention. Various therapeutic agents are produced by following the conventional approaches widely described in the literature. The various groups of therapeutic agents which can be envisaged in the present invention are described below.

As defined above, the therapeutic agent is capable of inhibiting or of decreasing, directly or indirectly, superantigen activity, as defined in the present invention, for one or more given Vβs and/or for one or more MSRV retroviruses. The therapeutic agent consists of a chemical or biological material or of a composition which has a therapeutic effectiveness which can be demonstrated according to the methods described above.

In the interests of simplicity of the presentation, the general expression “molecules of interest of the invention” will be used both to denote the various Vβ(s) associated with the superantigen activity and to denote the MSRV-1 proteins, in particular the env protein of MSRV-1.

The term “therapeutic agent” is intended to mean:

at least one natural molecule and/or one recombinant molecule, or fragments thereof, the sequence of which corresponds to all or part of the sequence of the molecules of interest, i.e.:

at least one natural protein and/or one recombinant protein and/or one synthetic polypeptide chosen from the molecules of interest of the invention,

at least one natural and/or synthetic fragment of these molecules of interest, for example an immunogenic fragment capable of inducing an immune response against a polypeptide derived from at least one of the molecules of interest of the invention,

at least one mimotope peptide defined on the basis of the sequences of the molecules of interest of the invention, or a combination of mimotopes, capable of inducing an immune response against a polypeptide derived from at least one of the molecules of interest of the invention,

at least one protein or one peptide which can regulate, in vivo, the transcription and/or the translation of the molecules of interest of the invention and the peptide sequences or the fragments of said sequences.

The immune response directed against a specific antigen can be divided into two different categories, one involving antibodies (immune response of the humoral type), the other involving cytotoxic effector cells such as, for example, macrophages, cytotoxic lymphocytes (CTLs) or killer (NK) cells, and also helper T lymphocytes, in particular CD4+ T lymphocytes (immune response of the cellular type). More particularly, the two types of response differ in that the antibodies recognize the antigens in their three-dimensional form, whereas the T lymphocytes, for example, recognize peptide portions of said antigens, associated with glycoproteins encoded by the genes of the major histocompatibility complex (MHC), in particular the genes of the type I major histocompatibility complex which are expressed ubiquitously at the surface of cells or the genes of the type II major histocompatibility complex which are expressed specifically at the surface of cells involved in antigen presentation (APCs). 1) According to a first aspect, the immune response of the cellular type is characterized in that the T cells of the CD4+ type (helper T cells), subsequent to a well known phenomenon of activation (for a review see Alberola-lia 1997, Annu Rev Immunol 15, 125-154) produce cytokines which, in turn, induce the proliferation of APCs capable of producing said cytokines, the cellular differentiation of B lymphocytes capable of producing antibodies specific for the antigen, and the stimulation of cytotoxic T lymphocytes (CTLs). 2) According to a second aspect of the cellular immune response, the cytotoxic effector cells, such as for example the lymphocytes of the CD8+ type (CTLs), are activated a) after interaction with antigenic peptides bound to and presented by the glycoproteins which are borne by ubiquitous cells and which are encoded by genes belonging to the MHCI system, and b) possibly by the cytokines produced by the CD4+ cells.

On the basis of the amino acid sequence of the molecules of interest of the invention, peptide sequences of these molecules or fragments of peptides sequences of these molecules, corresponding to all or part of the primary sequence of these molecules, can be synthesized using conventional methods of peptide synthesis or obtained by genetic recombination.

Recombinant proteins corresponding to the molecules of interest of the invention, produced in a prokaryotic or eukaryotic cell system, can be produced by those skilled in the art based on the knowledge of the sequences of the corresponding genes described in the literature and taking into account the degeneracy of the genetic code. All the protein sequences identified in the present invention can thus be obtained by genetic recombination. The genes are cloned in suitable vectors. Different vectors are used to transform prokaryotic cells (for example E. coli), and eukaryotic cells (for example COS cells, CHO cells and Simliki cells). The recombinant proteins corresponding to the molecules of interest of the invention or to fragments of these proteins can thus be produced in prokaryotic and/or eukaryotic cell systems. In E. coli cells, the recombinant proteins are produced with a polyhistidine tail. The insoluble protein fraction is solubilized in 8M urea. The product is enriched on resin chelated with nickel (Qiagen). The column is washed with decreasing concentrations of urea. Elution is carried out with imidazole in the absence of urea. The complete sequence of the molecules of interest of the invention may also be cloned into a suitable plasmid and then transferred into the vaccinia virus so as to obtain a recombinant virus;

at least one ligand specific for at least one of said molecules of interest of the invention, or of the fragments thereof, which is capable of binding to the molecules of interest or to the receptors for said molecules of interest, or alternatively of interfering in the binding of the molecule of interest to the antigen-presenting cell and of inhibiting the superantigen activity, i.e.:

at least one polyclonal or monoclonal antibody or antibody fragment specific for at least one of said molecules of interest of the invention, or for the fragments thereof. This antibody may or may not be a neutralizing antibody, i.e. may or may not be capable of inhibiting the activity of the protein of interest. The ligand may be chosen from any molecule or molecule fragment capable of binding to the molecules of interest, for example receptors, cofactors of these molecules, polyclonal antibodies or monoclonal antibodies capable of binding to the molecules of interest or to any fragment.

These antibodies are very useful in particular for enabling the use of therapeutic compositions, since they produce, for example, immune reactions directed specifically against immunodominant epitopes or against antigens which exhibit great variability. Patients are administered with either soluble neutralizing antibodies so as to inhibit their function or soluble specific antibodies so as to eliminate the peptide by formation of immune complexes. The invention describes the use of antibodies capable of specifically recognizing at least one molecule of interest described in the present invention, for the therapeutic treatment and/or monitoring of a disease, preferably multiple sclerosis. These antibodies are polyclonal antibodies and preferably monoclonal antibodies. Preferably, these antibodies inhibits the function of the protein by binding. The capacity of the antibody to bind specificly to the protein is analyzed with conventional described techniques, such as for example with ELISA or Western blot assays using the natural or synthetic immunogenic peptide or molecule. The antibody titer is then determined. The capacity of the antibody to neutralize the function of the protein can be analyzed by various means, for example by determining the decrease in the activity of the molecule or of the immunogenic peptide, in the presence of the antibody.

For example, the monoclonal antibodies directed against a molecule of interest of the invention or a part of this molecule are produced using conventional techniques used to produce antibodies against surface antigens. Mice or rabbits are immunized (i) either with the natural or recombinant protein of interest, (ii) or with any immunogenic peptide of this protein of interest, (iii) or with murine cells which express the protein or peptide of interest and MHCII molecules. The Balb/c murine line is the most commonly used. The immunogen may also be a peptide chosen from the peptides defined from the primary sequences of the molecules of interest. The proteins or peptides are coupled to keyhole limpet hemocyanin, abbreviated as peptide-KLH, as a support for its use in immunization, or coupled to human serum albumin, abbreviated as peptide-HSA. The animals are given an injection of peptide-KLH or of peptide-HSA, using complete Freund's adjuvant (IFA) The sera and the hybridoma culture supernatants derived from the animals immunized with each peptide are analyzed for the presence of anti-molecule antibodies with an ELISA assay using the initial molecules. The spleen cells of these mice are recovered and fused with myeloma cells. Polyethylene glycol (PEG) is the most commonly used fusion agent. The hybridomas producing the most specific and the most sensitive antibodies are selected. The monoclonal antibodies can be produced in vitro by cell culture of the hybridomas produced or by recovering murine ascites fluid after intraperitoneal injection of the hybridomas into mice. Whatever the method of production as supernatant or as ascites, it is then important to purify the monoclonal antibody. The purification methods used are essentially filtration over ion exchange gel or by exclusion chromatography, or even immunoprecipitation. For each antibody, the method which will make it possible to obtain the best yield should be chosen. A sufficient number of anti-protein antibodies are screened in functional assays in order to identify the antibodies which are the most effective in binding the molecule of interest and/or in blocking the activity of the molecule of interest. The monoclonal antibodies selected are humanized using standard “CDR grafting” methods (protocol carried out by many companies, in the form of a service). These humanized antibodies can be tested clinically in patients. The effectiveness of these antibodies can be monitored using clinical parameters.

The in vitro production of antibodies, of antibody fragments or of antibody derivatives, such as chimeric antibodies which may or may not be humanized and which are produced by genetic engineering, in eukaryotic cells has been described (EP 120 694 or EP 125 023) and can also be applied to the present invention,

at least one molecule which inhibits the function of at least one molecule chosen from the molecules of interest of the invention or the fragments thereof,

at least one molecule which regulates the expression of at least one molecule chosen from the molecules of interest of the invention or the fragments thereof, for example to block the transcription or the translation of these molecules,

at least one molecule which regulates the metabolism of at least one protein chosen from the molecules of interest of the invention or the fragments thereof,

at least one molecule which regulates the expression and/or the metabolism of a ligand for at least one protein chosen from the molecules of interest of the invention or the fragments thereof, for example a receptor or a cofactor,

at least one nucleic acid sequence comprising at least one gene of therapeutic interest, the nucleic acid sequence of which is deduced from the DNA and RNA sequences encoding all or part of the molecules of interest of the invention, in combination with elements which ensure the expression of said gene of therapeutic interest in vivo in target cells intended to be genetically modified with the nucleic acid sequence of the gene of therapeutic interest. The genes may or may not be mutated. They may also consist of nucleic acids modified such that it is not possible for them to integrate into the genome of the target cell, or of nucleic acids stabilized using agents such as spermine. Such a gene of therapeutic interest in particular encodes:

at least a protein chosen from the molecules of interest identified in the present invention or the fragments thereof, and/or

at least a ligand or any part of a ligand capable of attaching to at least one protein or one protein fragment chosen from the molecules of interest identified in the present invention or the fragments thereof, which may or may not inhibit the function of the molecule of interest, and/or

at least all or part of a polyclonal or monoclonal antibody capable of attaching to at least one protein or one protein fragment chosen from the molecules of interest identified in the present invention or the fragments thereof, which may or may not inhibit the function of the molecule of interest. It may in particular be a native transmembrane antibody, or a fragment or derivative of such an antibody, provided that said antibody, antibody fragment or antibody derivative is expressed at the surface of the genetically modified mammalian target cell and is capable of binding to a polypeptide present at the surface of a cytotoxic effector cell or of a helper T lymphocyte involved in the process of activation of such a cell, and/or

at least a molecule which inhibits at least one protein or the fragments thereof, said protein being chosen from the molecules of interest identified in the present invention, which can inhibit the function and/or the metabolism and/or the binding of the molecules of interest or of the fragments thereof.

Moreover, said nucleic acid may comprise at least two sequences, which may be identical or different, having transcriptional promoter activity and/or at least two genes, which may be identical or different, located, with respect to one another, contiguously, far apart, in the same direction or in opposite directions, provided that the transcriptional promoter function or the transcription of said genes is not affected.

Similarly, it is possible to introduce into this type of nucleic acid construct “neutral” nucleic acid sequences or introns which do not harm the transcription and are spliced before the translation step. Such sequences and the uses thereof are described in the literature (reference: PCT patent application WO 94/29471).

Said nucleic acid may also comprise sequences required for intracellular transport, for replication and/or for integration, for transcription and/or translation. Such sequences are well known to those skilled in the art.

Moreover, the nucleic acids which can be used according to the present invention may also be nucleic acids modified such that it is not possible to integrate them into the genome of the target cell, or nucleic acids stabilized using agents, such as for example spermine, which, per se, have no effect on the effectiveness of transfection.

The nucleic acid sequence is preferably a DNA or RNA sequence which is naked, i.e. free of any compound which facilitates its introduction into cells (nucleic acid sequence transfer). However, according to a second embodiment of the invention, in order to promote its introduction into target cells and in order to obtain the genetically modified cells of the invention, this nucleic acid sequence may be in the form of a “vector”, and more particularly in the form of a viral vector, such as for example an adenoviral vector, a retroviral vector or a vector derived from a poxvirus, in particular derived from the vaccinia virus or from the Modified Virus Ankara (MVA), or of a nonviral vector, such as for example a vector consisting of at least one said nucleic acid sequence complexed with or conjugated to at least one carrier molecule or substance selected from the group consisting of a cationic amphiphile, in particular a cationic lipid, a cationic or neutral polymer, a polar proctic compound, in particular chosen from propylene glycol, polyethylene glycol, glycerol, ethanol, 1-methyl-L-2-pyrrolidone, or derivatives thereof, and a polar aprotic compound, in particular chosen from dimethyl sulfoxide (DMSO), diethyl sulfoxide, di-n-propyl sulfoxide, dimethylsulfone, sulfolane, dimethylformamide, dimethylacetamide, tetramethylurea, acetonitrile, or derivatives thereof The literature provides a considerable number of examples of such viral and nonviral vectors.

Such vectors may also and preferably comprise targeting elements which may make it possible to direct the nucleic acid sequence transfer toward certain cell types or certain particular tissues, such as helper T cells, cytotoxic T cells and antigen-presenting cells. They may also make it possible to direct the transfer of an active substance toward certain preferred intracellular compartments, such as the nucleus, the mitochondria or the peroxisomes, for example. It may also involve elements which facilitate penetration into the cell or lysis of intracellular compartments. Such targeting elements are widely described in the literature. They may, for example, be all or a part of lectins, of peptides, in particular the JTS-1 peptide (see PCT patent application WO 94/40958), of oligonucleotides, of lipids, of hormones, of vitamins, of antigens, of antibodies, of ligands specific for membrane-bound receptors, of ligands capable of reacting with an anti-ligand, of fusogenic peptides, of peptides with a nuclear location, or of a composition of such compounds,

or alternatively,

at least one nucleic acid sequence capable of hybridizing to a nucleic acid sequence encoding the molecules of the interest of the invention or the fragments thereof. The fragments correspond in particular to antisense or ribozyme molecules and can be synthesized using automatic synthesizers, such as those sold by the company Applied Biosystems. Antisense oligonucleotides are capable of interfering specifically with the synthesis of a target protein of interest, by inhibiting the formation and/or the functioning of the polysome according to the position of the mRNA in the target. Therefore, the frequent choice of the sequence surrounding the translation initiation codon as the target for inhibition with an antisense oligonucleotide is directed toward preventing the formation of the initiation complex. Other mechanisms in the inhibition with antisense oligonucleotides involve activation of ribonuclease H, which digests the antisense oligonucleotide/mRNA hybrids, or interference at the splice sites with antisense oligonucleotides, the target of which is an mRNA splice site. Antisense oligonucleotides are also complementary to DNA sequences and can therefore interfere with transcription by forming a triple helix, the antisense oligonucleotide pairing via “Hoogsteen” hydrogen bonds at the level of the major groove of the DNA double helix. In this particular case, the term “anti-gene oligonucleotide” is more precisely used. It is clearly understood that antisense oligonucleotides may be strictly complementary to the DNA or RNA target to which they must hybridize, but also not strictly complementary, on the condition that they hybridize to the target. Similarly, they may be antisense oligonucleotides in which the internucleotide bonds may or may not be modified. All these notions form part of the general knowledge of those skilled in the art. Of course, such antisense molecules may constitute vectors, per se. Use may also be made of vectors which comprise a nucleic acid sequence which encodes an antisense. These nucleic acid molecules are capable of hybridizing, under stringent conditions, to the DNA and/or RNA encoding the proteins of the invention or the fragment(s) thereof. Characteristic conditions of stringency are those which correspond to a combination of the temperature and of the saline concentration chosen approximately between 12 and 20° C. under the Tm (melting temperature) of the hybrid being studied. Such molecules are synthesized and can be labeled using conventional labeling methods used for molecular probes, or can be used as primers in amplification reactions. The sequences which show at least 90% homology compared to a reference sequence also form part of the invention, as do the fragments of these sequences which have at least 20 contiguous nucleotides, and preferably 30 contiguous nucleotides, which are homologous compared to a reference sequence.

The DNA or RNA nucleic acid sequences or molecules consist of a double-stranded or single-stranded, linear or circular, natural and isolated or synthetic DNA and/or RNA fragment corresponding to a precise chain of nucleotides, which may or may not be modified, and which make it possible to define a fragment or a region of a nucleic acid chosen from the group consisting of a cDNA; a genomic DNA; a plasmid DNA and a messenger RNA. The nucleic acid sequences are deduced from the amino acid sequence of the molecules of interest of the invention or of the fragments thereof, using the genetic code. Because of the degeneracy of the genetic code, the invention also encompasses equivalent or homologous sequences. These defined sequences allow those skilled in the art to, themselves, define the suitable molecules. They can thus define and use nucleic acid molecules which are complementary to the DNA and/or RNA sequences encoding the molecules of interest of the invention or to the fragment(s) thereof.

These nucleic acid sequences and/or vectors make it possible to target the cells in which the protein or the protein fragment is expressed, either using a targeting molecule introduced onto the vector or using a particular property of the cell;

at least one mammalian cell which does not naturally produce at least one molecule of interest of the invention or any fragment of these molecules, or antibodies specific for at least one of said molecules of interest of the invention or of the fragments thereof, said mammalian cell being genetically modified in vitro with at least one nucleic acid sequence or a fragment of a nucleic acid sequence or a combination of nucleic acid sequences corresponding to nucleic acid fragments derived from the same gene or from different genes, said nucleic acid sequence(s) being deduced from the DNA and RNA sequences encoding the molecules of interest of the invention or any fragment, said gene of therapeutic interest encoding all or part of the molecule of interest of the invention, of a fragment of the molecule or of an antibody specific for the molecule which will be expressed at the surface of said mammalian cell (Toes et al., 1997, PNAS 94: 14660-14665). Thus, said cell contains at least one gene which encodes in vivo:

at least one protein chosen from the molecules of interest of the invention and/or the fragments thereof, and/or

at least one peptide defined on the basis of the primary sequence of at least one protein chosen from the molecules of interest of the invention and/or the fragments thereof, and/or

at least any molecule which inhibits the activity and/or the binding and/or the expression of these molecules, and/or

at least one peptide derived from the primary sequence of a protein chosen from the molecules of interest of the invention and/or the fragments thereof, and capable of binding to at least one MHCI and/or MHCII glycoprotein, and/or

at least one ligand and/or any antibody and/or any part of an antibody capable of binding to at least one protein chosen from the molecules of interest of the invention and/or the fragments.

More particularly, said target cell originates either from the mammal to be treated or from a mammal other than the one to be treated. In the latter case, it should be noted that said target cell will have undergone a treatment making it compatible with the mammal to be treated. According to a preferred aspect, the term “mammal” is intended to denote a human mammal. These cells are established as cell lines and are preferentially MHCII+ or MHCII− inducible such as lymphocytes, monocytes, astrocytes, oligodendrocytes.

The invention also relates to the modified cells and a method for preparing a cell as described above, characterized in that at least one nucleic acid sequence containing at least one gene of therapeutic interest and elements which ensure the expression of said gene in said cell are introduced into a mammalian cell by any suitable means, said gene of therapeutic interest containing a nucleic acid sequence encoding a molecule or a molecule fragment in vivo, as described just above. More particularly, it relates to prokaryotic cells, yeast cells and animal cells, in particular mammalian cells, transformed with at least one nucleotide sequence and/or one vector as described above.

According to a particular embodiment, the cells (dendritic cells, macrophages, astrocytes, CD4+ T lymphocytes, CD8+ T lymphocytes) of the patient or allogenic cells are placed in contact with a purified preparation of the target polypeptide, the latter being internalized, processed and presented at the cell surface, associated with MHCI and/or MHCII molecules. They may, inter alia, induce a specific immune response against the peptide. The “activated” cells are then administered to the patient, in which they may, inter alia, induce an immune response specific for the antigens (a natural pathway of the immune response is used, but control is exerted over that which the cell presenting the antigen will present).

According to a particular embodiment, the antigen-presenting cells (dendritic cell, macrophage, astrocytes, etc.) are modified in vitro so as to express the antigens in the transformed cell, which can be secreted by the cell and/or associate with MHCI and/or MHCII molecules and be presented at the surface of the cells so as to induce, in the patient to which the modified cell is administered, a perfectly targeted immune reaction.

Not all immunization approaches are always satisfactory and produce, for example, limited immune reactions directed only against immunodominant epitopes or against antigens exhibiting great variability. Similarly, the incorrect presentation of antigens by the glycoproteins of the MHC system at the surface of cells does not make it possible to develop suitable anti-protein of interest immunity in the patient treated. In order to overcome these problems, certain authors have proposed, in the context of such immunization methods, selecting the minimum antigenic fragments corresponding to the peptide portions capable of being recognized specifically by cytotoxic T lymphocytes, and expressing them in cells so that they associate with MHCI molecules and are presented at the surface of the cells so as to induce, in the patient treated, a perfectly targeted immune reaction (Toes et al. 1997, PNAS 94: 14660-14665). More particularly, it has been shown that epitopes which are very small in size (ranging from 7 to approximately 13 amino acids) and which are expressed from minigenes introduced into a vaccinia virus can induce an immunization of the cellular type. It has, moreover, been shown that several minigenes can be expressed jointly using the same vector (this particular construct is termed “string of beads”). Such a construct has the advantage of inducing a synergistic immune reaction of the CTL type (Whitton et al., 1993 J. of Virology 67: 348-352).

Protocol for bringing the cells and the antigenic fragment into contact:

The presentation of antigenic fragments by MHCI and/or MHCII molecules is based on an identified intracellular process (see Groettrup et al., 1996 Immunology Today 17: 429-435 for a review) during which antigenic peptides which are very small in size (approximately 7-13 amino acids) are produced by degradation of a more complex polypeptide against which the final immune reaction will be directed. These short peptides are then associated with MHCI or MHCII molecules so as to form a protein complex which is transported to the cell surface in order to present said peptides to circulating cytotoxic T lymphocytes or to circulating helper T lymphocytes, respectively. It should also be noted that the specificity of the MHCI or MHCII molecules with respect to the antigenic peptides varies as a function of the MHCI or MHCII molecules (example for MHCI: HLA-A, HLA-B, etc.) and of the allele (example for MHCI: HLA-A2, HLA-A3, HLA-A11) under consideration. Within the same animal species, from one individual to the other, there exists a great variability of the genes encoding the molecules of the MHC system (in this respect, see in particular George et al., 1995, Imunology Today 16: 209-212).

According to a particular embodiment, the cells, such as dendritic cells, macrophages, astrocytes, CD4+ T lymphocytes, CD8+ T lymphocytes, are modified so as to express at their surface antibodies specific for the targeted peptide. The peptide is neutralized by the antibodies expressed at the surface of the cells. These cells are preferably immune cells, preferably from the patient, preferably cytotoxic, modified so as to express all or part of an antibody specific for the target polypeptide.

Isolation of mononucleated cells from peripheral blood:

In 1968, Boyum described a rapid technique which makes it possible, by centrifuging blood on a density gradient, to separate the mononucleated cells (lymphocytes and monocytes) with a good yield (50% theoretical yield, i.e. 106 cells/ml of blood). 50 ml of peripheral blood taken sterilely in heparinized tubes are centrifuged for 20 minutes at 150 g at 20° C. The cells recovered are diluted in two initial peripheral blood volumes of sterile PBS. 10 ml of this suspension are deposited onto 3 ml of a ficoll-Hypaque solution (lymphocyte separation medium, Flow). After centrifugation for 20 minutes at 400 g and 20° C. without deceleration brakeing, the mononucleated cells sediment at the PBS-Ficoll interface, in a dense opalescent layer, whereas virtually all of the red blood cells and polynuclear cells sediment at the bottom of the tube. The mononucleated cells are recovered and washed in sterile PBS.

Internalization of the antigens by the antigen-presenting cells:

Prior treatment of the antigen-presenting cells: the antigen-presenting cells are prewashed with a PBS/0.5% BSA (w/v) buffer then numbered, and are then preincubated in the presence of various reduction inhibitors three times, in PBS/0.5% BSA containing from 10 μM to 10 mM final of DTNB (5,5′-dithiobis-2-nitrobenzoic acid) or of NEM (N-ethylmaleimide). The subsequent steps of antigen binding to the cell surface or of antigen internalization also take place in the presence of the various concentrations of inhibitors.

Protocol for antigen internalization by the antigen-presenting cells:

8×10⁶ cells are incubated in the presence of a saturating amount of proteins radiolabeled with iodine 125 (1 μg), in microwells in 70 μl. After incubation for one hour at 4° C. with stirring, the antigens are bound to the surface of the cells. The cell suspension is washed twice in PBS/BSA and the cell pellets are taken up in 70 μl of buffer and incubated at 37° C. for various periods of time ranging up to 2 hours. Cells and supernatants are separated by centrifugation at 800 g for 5 minutes, 4° C. For longer periods of incubation, the preliminary step of prebinding of the antigens to the surface of the cells is deleted. The cells are diluted, in an RPMI/10% SVF medium in the presence of 20 mM Hepes, to 10⁶ cells/ml. The cells are incubated in the presence of an excess of antigen for various periods of time at 37° C. (1 μg of molecules/5×10⁷ monocyte/macrophage cells or /10⁸ B-EBV cells).

Using the knowledge of the amino acid sequences of the proteins of interest of the invention, it is within the scope of those skilled in the art to define and use the molecules described above and/or any molecule capable of binding to said molecules and/or any molecule capable of inhibiting the activity of said molecules of interest.

The therapeutic agent is preferably chosen from a natural molecule and/or a recombinant molecule, or a fragment of said molecules, the protein sequence of which corresponds to the sequence of the Vβ16 and/or Vβ17 molecules, preferably the Vβ16 molecule, optionally in combination with one or more natural and/or recombinant molecules, or a fragment of said molecules, the protein sequence of which corresponds to the sequences of the Vβ2, Vβ3, Vβ7, Vβ8, Vβ12, Vβ14, Vβ17 and Vβ22 molecules, and preferentially of the Vβ3 and Vβ12 molecules or of the Vβ7, Vβ14 and Vβ17 molecules, and advantageously the Vβ7 and Vβ17 molecules;

and from the natural and/or the recombinant and/or synthetic molecules, or a fragment of said molecules, which encode the molecules as defined above.

In particular, the therapeutic and/or prophylactic agent is chosen from DNA and/or RNA molecules; antisense oligonucleotides and anti-gene oligonucleotides; at least one ligand capable of interacting with Vβ16 and/or Vβ17, in particular Vβ16, optionally in combination with at least one ligand capable of interacting with at least one of Vβ2, Vβ3, Vβ7, Vβ8, Vβ12, Vβ14, Vβ17 and Vβ22, and preferentially Vβ3 and Vβ12; from antibodies, preferably monoclonal antibodies and anti-receptors for the TCRs of the various Vβs above; at least one ligand capable of interacting with Vβ16 and/or Vβ17, optionally in combination with at least one ligand capable of interacting with at least one of Vβ7, Vβ14, Vβ17 and Vβ22, and preferentially Vβ7 and Vβ17, in particular antibodies, and preferably monoclonal antibodies, or fragments of said antibodies and anti-receptors for the TCRs of the various Vβs above; an agent capable of blocking the interaction of the superantigen with the antigen-presenting cells; at least one cell, preferably a cell of mammalian origin, genetically modified in vitro with a therapeutic agent which consists of at least one nucleic acid molecule encoding at least one molecule, the protein sequence of which corresponds to the sequence encoding the molecules as defined above, in particular a DNA and/or RNA molecule; at least one cell, preferably a cell of mammalian origin, genetically modified in vitro with a therapeutic agent which consists of at least one nucleic acid molecule encoding at least one ligand as defined above, in particular a DNA and/or RNA molecule.

The therapeutic agent is preferably chosen

from a natural molecule and/or a recombinant molecule, or a fragment of said molecules, the protein sequence of which corresponds to the sequence of the MSRV-1 proteins, and advantageously to the sequence of the env protein of MSRV-1, and

from the natural and/or recombinant and/or synthetic molecules, or a fragment of said molecules, which encode the molecule as defined above.

In particular, the therapeutic and/or prophylactic agent is chosen from DNA and/or RNA molecules; antisense oligonucleotides and anti-gene oligonucleotides; at least one ligand capable of interacting with MSRV-1 proteins, in particular the env protein of MSRV-1, from antibodies, preferably monoclonal antibodies and anti-MSRV-1 proteins, in particular anti-env proteins of MSRV-1; at least one cell, preferably a cell of mammalian origin, genetically modified in vitro with a therapeutic agent which consists of at least one DNA molecule encoding at least one molecule, the protein sequence of which corresponds to the sequence encoding the molecules as defined above, in particular a DNA and/or RNA molecule; at least one cell, preferably a cell of mammalian origin, genetically modified in vitro with a therapeutic agent which consists of at least one DNA molecule encoding at least one ligand as defined above, in particular a DNA and/or RNA molecule.

Thus, the therapeutic agent is preferably an antiviral agent, more particularly an antiretroviral agent, in particular a human antiretroviral agent, preferably an anti-MSRV1 agent, such as an inhibitor of the replication cycle and/or of the expression of a retrovirus, such as an anti-retroviral protein antibody, in particular an anti-envelope antibody, such as antisense oligonucleotides, more particularly which block retroviral expression.

Advantageously, the ligand is capable of interacting with a retrovirus, in particular a human retrovirus, such as MSRV-1, the proteins thereof and/or the nucleic acids thereof. In particular, the ligand is chosen from anti-MSRV-1 antibodies, preferably monoclonal antibodies.

The therapeutic agents as defined above are used for preparing a prophylactic and/or therapeutic composition, and the invention also relates to such a composition comprising at least one of said therapeutic agents in optional combination with a pharmaceutically acceptable vehicle and/or excipient and/or adjuvant and/or diluent. They may be, inter alia, immunization compositions based on protein molecule(s) or on peptide(s) or on sequences of nucleic acids derived from the molecules of interest or from the fragments thereof, as described above, for the prophylaxis and/or the therapy of an autoimmune disease, in particular multiple sclerosis. Antiviral agents may be included among these molecules. These administered proteins and peptides are characterized in that they must not be toxic for the organism to which they are administered, or must have lost their capacity to bind to a ligand, and can significantly induce an immune response mediated by T lymphocytes and/or antibodies directed against this protein. Such proteins are termed “modified”, however, their immunogenicity is conserved. Such modified immunogenic molecules are obtained using a number of conventional treatments, for example chemical or heat denaturation, or truncation or mutation with deletion, insertion or replacement of amino acids. An example of truncation consists of the truncation of amino acids at the carboxy-terminal end which can range up to 5-30 amino acids. The modified molecules can be obtained using synthetic and/or recombinant techniques or using chemical or physical treatments of natural molecules.

The natural and/or recombinant molecules of interest identified in the present invention and the peptide sequences or the fragments of said molecules are used in prophylactic and therapeutic immunization against diseases, preferably multiple sclerosis. A vaccine comprises an effective immunogenic amount of the immunogenic molecule in combination with a pharmaceutically acceptable vehicle and, optionally, an adjuvant and/or a diluent. The pharmaceutically acceptable vehicles, adjuvants and diluents are well known to those skilled in the art. By way of reference, mention may be made of Remington's Pharmaceutical Sciences. The use of immunization compositions is particularly advantageous in combination with an early diagnosis of the disease. The immunogenic protein is used in the medicinal product preparation for the prophylactic or therapeutic immunization. The proteins of interest can be eliminated from the organism without inducing undesirable side effects. The identification of such vaccine peptides or molecules is carried out in the following way: it is verified that the modified candidate molecules as described above (natural proteins, recombinant proteins, peptides) are not toxic for the organism, and then their immunogenicity is verified (i) by carrying out an in vitro proliferation assay for CD4+ T lymphocytes specific for the antigen administered (T-cell assay) or an in vitro cytotoxicity assay for the CD8+ lymphocytes specific for the antigen administered and (ii) by measuring, inter alia, the level of circulating antibodies directed against the natural protein. These modified forms are used to immunize humans using standard procedures with suitable adjuvants.

The vaccines prepared are injectable, i.e. in liquid solution or in suspension. As an option, the preparation may also be emulsified. The antigenic molecule may be mixed with excipients which are pharmaceutically acceptable and compatible with the active ingredient. Examples of favorable excipients are water, a saline solution, dextrose, glycerol, ethanol or equivalents and combinations thereof. If desired, the vaccine may contain minor amounts of auxiliary substances such as wetting agents or emulsifiers, pH-buffering agents or adjuvants such as aluminum hydroxide, muramyl dipeptide or variations thereof. In the case of peptides, the coupling thereof to a larger molecule (KLH, tetanus toxin) sometimes increases the immunogenicity. The vaccines are administered conventionally by injection, for example subcutaneous or intramuscular injection. Additional formulations which are favorable with other modes of administration include suppositories and sometimes oral formulations.

In general, the protein concentration in the composition used for in vivo administration is, for example, from 0.1 μg/ml to 20 mg/ml.

The present invention also relates to the use of vaccines including nucleic acid molecules which encode the molecules of interest of the invention or immunogenic peptides or the fragment(s) thereof, which are nonactive. The nucleic acid vaccines, in particular the DNA vaccines, are generally administered in combination with a pharmaceutically acceptable vehicle, by intramuscular injection.

Another aspect of the invention relates to prophylactic and/or therapeutic compositions which comprise, inter alia, natural and/or synthetic and/or recombinant substances (i) capable of blocking and/or of inhibiting the activity of the molecules of interest of the invention and/or of the fragments thereof, and/or (ii) capable of inhibiting their metabolism, and/or (iii) capable of regulating the expression of the molecules of interest of the invention or the fragments thereof, and/or (iv) capable of inhibiting the function and/or the expression of the ligands for the molecules of interest of the invention or the fragments thereof. These substances may be used in prophylactic and therapeutic treatments for the disease, for example MS, by administering effective amounts. The substances may be small synthetic or natural molecules, derivatives of the proteins identified in this invention, lipids, glycolipids, chemical compounds, etc. The small molecules can be screened and identified in large amount by using chemical combinatorial libraries. The invention also relates to pharmaceutical compositions comprising these substances in combination with physiologically acceptable vehicles and/or adjuvants and/or excipients and/or diluents, and methods for preparing medicinal products to be used in the therapy or in the prevention of autoimmune diseases, including MS, using these substances.

In order to identify such molecules, use is made of the in vitro and in vivo assays and protocols as described above, using samples taken from untreated or treated patients and/or animals. The molecules selected are tested at various concentrations. These inhibitors are also tested in pharmacokinetics and toxicity assays in order to determine whether they may represent valid candidate drugs. The small molecules can be screened and identified in large amount using chemical combinatorial libraries.

The present invention also relates to the use of cells transformed in vivo after the injection of vectors containing at least one gene of therapeutic interest defined on the basis of the molecules of interest identified or the fragments thereof, for preparing a prophylactic and/or therapeutic “gene therapy” composition, and said composition. It is recalled that, in the interest of simplicity of the presentation, the general expression “molecules of interest of the invention” will be used to denote the various Vβ(s) associated with the superantigen activity and the MSRV-1 protein(s), in particular the env protein of MSRV-1.

The composition comprises at least one vector containing a therapeutic gene as described below, capable of being introduced into a target cell in vivo and of expressing the therapeutic gene of interest in vivo. The advantage is based on the possibility of maintaining, over the long term, a basal level of molecules expressed in the patient treated. Vectors or nucleic acids encoding genes of therapeutic interest are injected. These vectors and nucleic acids must be transported to the target cells and transfect these cells in which they must be expressed in vivo.

The invention relates to the in vivo expression of nucleotide sequences and/or of vectors as described above, i.e. sequences corresponding to genes of therapeutic interest, in particular:

either at least encoding a protein chosen from the molecules of interest identified in the present invention or the fragments thereof; and/or

at least encoding all or part of a polyclonal or monoclonal antibody capable of binding to at least one protein chosen from the molecules of interest identified in the present invention and the fragments thereof. It may be a native transmembrane antibody, or a fragment or derivative of such an antibody, provided that said antibody, antibody fragment or antibody derivative is expressed at the surface of the genetically modified mammalian target cell and that said antibody is capable of binding to a polypeptide present at the surface of a cytotoxic effector cell or of a helper T lymphocyte and inhibiting the activity of at least one molecule of interest of the invention. They may be antibody fragments expressed by cells capable of secreting said antibodies into the blood circulation of a mammal or patient carrying the cells genetically modified with the gene encoding the antibody; and/or

at least encoding a molecule which inhibits at least one protein chosen from the molecules identified in the present invention or the fragments thereof; and/or

at least encoding a ligand or any part of the ligand capable of binding to at least one protein chosen from the molecules of interest identified in the present invention or the fragments thereof, and/or of inhibiting its function. Using the amino acid sequences of the molecules of interest of the invention or of the fragments thereof, it is within the scope of those skilled in the art to deduce the DNA and RNA nucleotide sequences corresponding to the molecules of interest or to the fragments thereof, using the genetic code and taking into account the degeneracy thereof. Thus, the present invention relates to the use of these nucleotide sequences in the form of antisense sequences, of sequences encoding a therapeutic gene and of sequences which can be contained in a vector for performing cell transformation ex vitro and/or in vivo (gene therapy).

According to a particular embodiment, it involves using gene therapy so as to direct the immune response against the target protein, peptide or molecule of interest, i.e. against any molecule chosen from the molecules of interest of the invention and/or the fragments thereof, and/or against any molecule which inhibits the activity and/or the expression and/or the metabolism of said molecules of interest, and/or ligands for said molecules. For this, it is evident that the cells to be targeted for transformation with a vector are cells belonging to the immune system, either antigen-presenting cells (dendritic cells, macrophages, etc.) or cells of the lymphocyte type (CD4/CD8).

According to a particular embodiment, the antigen-presenting cells (APCs) are genetically modified, in particular in vivo. APCs such as macrophages, dendritic cells, microgliocytes or astrocytes play a role in initiating the immune response. They are the first cellular components which capture the antigen, process it in the cell and express transmembrane MHCI and MHCII molecules involved in presenting the immunogen to CD4+ and CD8+ T cells; they produce specific secondary proteins which contribute to the activation of the T cells via the recognition of the Vβ determinants of the T receptor at the surface of the T lymphocytes (Debrick et al., 1991, J. Immunol 147: 2846; Reis et al., 1993, J Ep Med 178: 509; Kovacsovics-bankowski et al., 1993, PNAS 90: 4942; Kovacsovics-bankowski et al., 1995 Science 267: 243; Svensson et al., 1997, J Immunol 158: 4229; Norbury et al., 1997, Eur J Immunol 27: 280). For immunization, it may be advantageous to have a gene therapy system which can target the gene transfer into such APCs.

It is chosen to express, at the surface of the APCs in vivo, all or part of an antibody, and/or a ligand, capable of reacting with the target protein or peptide chosen from the molecules of interest of the invention and/or the fragments thereof. Such cells will then specifically phagocytose said protein or said peptide and process it so that fragments of this peptide are presented at the surface of the antigen-presenting cells.

The literature provides a large number of examples of genes encoding antibodies capable of reacting with polypeptides or receptors. It is within the scope of those skilled in the art to obtain the nucleic acid sequences encoding such antibodies. Mention will be made, for example, of the genes encoding the light and heavy chains of the YTH 12.5 (anti-CD3) antibody (Routledge et al. 1991, Eur J Immunol 21: 2717-2725) and of the anti-CD3 according to Arakawa et al; 1996, J. Biochem. 120: 657-662. The nucleic acid sequences of such antibodies can be easily identified from the databases commonly used by those skilled in the art. It is also possible, using hybridomas available from the ATCC, to clone the nucleic acid sequences encoding the heavy and/or light chains of these various antibodies using amplification methods such as RT-PCR with the aid of specific oligonucleotides, or techniques using cDNA libraries (Maniatis et al., 1982, Molecular cloning. A laboratory manual CSH Laboratory, Cold Spring Harbor, N.Y.). The sequences thus cloned are then available to be cloned into vectors. According to a preferred case of the invention, the nucleic acid sequence encoding the heavy chain of the antibody is fused by homologous recombination with a nucleic acid sequence encoding a transmembrane polypeptide (Polydefkis et al., 1990 J Exp Med 171: 875-887). These molecular biology techniques have been very well described.

It is chosen to express, at the surface of the APCs in vivo, immunogenic fragments corresponding to at least one protein chosen from the molecules of interest of the invention and/or the fragments thereof. For this, it may be chosen to have the vector express either the complete polypeptide or, preferably, polypeptides selected to react with specific ligands and/or receptors. The immunogenic peptide encoded by the polynucleotide introduced into the cell of vertebrate in vivo may be produced and/or secreted, processed and then presented to an antigen-presenting cell (APC) in the context of MHC molecules. The APCs thus undergoing transfer in vivo induce an immune response directed against the immunogen expressed in vivo. APCs have various mechanisms for capturing antigens: (a) capture of antigens by membrane-bound receptors such as immunoglobulin (Fc) receptors or receptors for complement available at the surface of granulocytes, monocytes or macrophages which allow effective delivery of the antigen into intracellular compartments after receptor-mediated phagocytosis; (b) entry into the APCs by fluid phase pinocytosis involving various mechanisms: micropinocytosis, i.e. the capture of small vesicles (0.1 μm) by clathrin-coated pits and macropinocytosis, i.e. the capture of larger vesicles (with a size ranging between 0.5 μm and approximately 6 μm) (Sallusto et al. 1995, J Exp Med 182: 389-400). While micropinocytosis exists constitutively in all cells, macropinocytosis is limited to cell types such as, for example, macrophages, dendritic cells, astrocytes or epithelial cells stimulated by growth factors (Racoosin et al., J Cell Sci 1992, 102: 867-880). In this invention, the expression “cells capable of macropinocytosis” is intended to mean cells which can perform the events described above and cells which can capture macromolecules preferably between 0.5 μm and approximately 6 μm in the cytoplasm.

According to a particular embodiment, cytotoxic effector cells or helper T lymphocytes are genetically modified, in particular in vivo, so that they express, at their surface, ligands for at least one of said molecules of interest of the invention, which are not naturally expressed by these cells, and which are capable of binding to all or part of at least one of the molecules of interest of the invention at the surface of the same cell or of another cell, and of inhibiting the activity of at least one molecule of interest of the invention, by introducing into these cells nucleic acid sequences containing the gene encoding such a polypeptide. In accordance with the present invention, it is also possible to select a nucleic acid sequence containing a gene of therapeutic interest encoding all or part of an antibody directed against a protein chosen from the molecules of interest of the invention and the peptide sequences and/or the fragments of said sequences, which is capable of being expressed at the surface of the target cells of the patient to be treated, said antibody being capable of binding, via these effector cells, to a polypeptide of the molecules of interest of the invention present at the surface of the cytotoxic lymphocytes and/or helper T lymphocytes, or even of inhibiting the activity of these molecules of interest.

Many tools have been developed for introducing various heterologous genes and/or vectors into cells, in particular mammalian cells. These techniques can be divided into two categories. The first category involves physical techniques such as microinjection, electroporation or particle bombardment. The second category is based on the use of molecular and cell biology techniques with which the gene is transferred with a biological or synthetic vector which facilitates the introduction of the material into the cell in vivo. Today, the most effective vectors are viral vectors, in particular adenoviral or retroviral vectors. These viruses have natural properties for crossing plasma membranes, avoiding the degradation of their genetic material and introducing their genome into the nucleus of the cell. These viruses have also been widely studied and some are already used experimentally in human applications in immunization, in immunotherapy or for compensating for genetic deficiencies. However, this viral approach has limitations in particular due to the restricted cloning capacity in these viral genomes, the risk of disseminating the viral particles produced in the organism and the environment, the risk of artefactual mutagenesis by insertion into the host cell in the case of retroviruses and the possibility of inducing a strong inflammatory immune response in vivo during treatment, which limits the number of possible injections (McCoy et al. 11995, human Gene Therapy 6: 1553-1560; Yang et al., 1996 Immunity 1: 433-422). Other systems alternative to these viral vectors exist; the use of nonviral methods such as, for example, coprecipitation with calcium phosphate, the use of receptors which mimic viral systems (for a summary see Cotten and Wagner 1993, Current Opinion in Biotechnology, 4: 705-710) or the use of polymers such as polyamidoamines (Haensler and Szoka 1993, Bioconjugate Chem 4: 372-379). Other nonviral techniques are based on the use of liposomes, the effectiveness of which for introducing biological macromolecules such as DNA, RNA, proteins or active pharmaceutical substances has been widely described in the scientific literature. In this field, teams have proposed the use of cationic lipids with a high affinity for cell membranes and/or nucleic acids. In fact, it has been shown that a nucleic acid molecule can, itself, cross the plasma membrane of certain cells in vivo (WO 90/11092), the effectiveness being dependent in particular on the polyanionic nature of the nucleic acid. From 1989 (Felgner et al., Nature 337: 387-388), cationic lipids were proposed for facilitating the introduction of large anionic molecules which neutralizes the negative charges of these molecules and promotes their introduction into cells. Various teams have developed such cationic lipids: DOTMA (Felgner et al., 1987, PNAS 84: 7413-7417), DOGS or Transfectam™ (Behr et al., 1989, PNAS 86: 6982-6986), DMRIE and DORIE (Felgner et al., 1993 methods 5: 67-75), DC-CHOL (Gao and Huang 1991, BBRC 179: 280-285), DOTAP™ (McLachlan et al., 1995, Gene therapy 2: 674-622) or Lipofectamine™, and the other molecules described in patents WO 91/16024, WO 95/14651 and WO 94/05624. Other groups have developed cationic polymers which facilitate the transfer of macromolecules, in particular anionic macromolecules, into cells. Patent WO 95/24221 describes the use of dendritic polymers, document WO 96/02655 describes the use of polyethyleneimine or polypropyleneimine and documents U.S. Pat. No. 5595897 and FR 2719316 describe the use of polylysine conjugates.

Given that the intention is to obtain a transformation targeted toward a given cell type, in vivo, it is evident that it must be possible for the vector used to itself be “targeted” (as described above).

The biological material defined in the present invention may be administered in vivo, in particular in injectable form. Epidermal, intravenous, intra-arterial, intramuscular, and intracerebral injection by syringe or any other equivalent means may also be envisaged; according to another embodiment, it may be administered orally or by any other means entirely known to those skilled in the art and applicable to the present invention. The administration may take place as a single dose or a dose repeated one or more times after a certain period of delay. The most suitable route of administration and dose vary as a function of various parameters such as, for example, the individual or the disease to be treated, of the stage and/or of the evolution of the disease, or alternatively of the nucleic acid and/or of the protein and/or peptide and/or molecule and/or cell to be transferred or of the target organ/tissue.

Other subjects of the invention are as follows:

a method for identifying substances capable of blocking the transcription and/or the translation of a human retrovirus, in particular a retrovirus which is endogenous, as defined above, and which has superantigen activity, said superantigen activity being associated with an autoimmune disease, according to which,

the substance is brought into contact with cells expressing a retroviral polypeptide as defined above which has superantigen activity, and

a loss or decrease of the superantigen activity is detected according to the invention;

a kit for screening substances capable of blocking the superantigen activity of a retrovirus, in particular an endogenous human retrovirus, associated with an autoimmune disease, or capable of blocking the transcription and/or the translation of said retrovirus, comprising:

cells expressing, at their surface, class II MHC products, transformed with and functionally expressing a retroviral superantigen,

cells bearing receptor chains for one or more Vβs stimulated by the retroviral superantigen, and

means for detecting a loss or decrease of the superantigen activity according to the invention;

the use of substances capable of inhibiting a function of a human retrovirus, in particular an endogenous retrovirus, for preparing a medicinal product for use in therapy and/or prevention of an autoimmune disease associated with a retroviral superantigen, in particular associated with S, advantageously the substances are chosen from AZT and DDI (dideoxyinosine);

the use of substances capable of inhibiting the superantigen function of a human retrovirus, in particular an endogenous retrovirus, for preparing a medicinal product for use in the therapy of an autoimmune disease associated with a retroviral superantigen, in particular associated with MS.

Definitions:

The term “antibody fragment” is intended to mean the F(ab)2, Fab′, Fab and sFv fragments (Blazer et al., 1997, Journal of Immunology 159: 5821-5833; Bird et al., 1988 Science 242: 423-426) of a native antibody, and the term “derivative” is intended to mean, for example, a chimeric derivative of such an antibody (see, for example, the mouse/human anti-CD3 antibody chimeras in Arakawa et al., 1996 J Biochem 120: 657-662 or the immunotoxins such as sFv-toxin of Chaudary et al. 1989, Nature 339: 394-397).

The term “transmembrane antibody” is intended to mean an antibody in which at least the functional region capable of recognizing and of binding to its specific antigen is expressed at the surface of target cells so as to allow said recognition and binding. More particularly, the antibodies according to the present invention consist of fusion polypeptides comprising the amino acids which define said functional region and an amino acid sequence (transmembrane polypeptide) which allows anchoring in the lipid bilayer of the membrane of the target cell or at the external surface of this bilayer. The nucleic acid sequences encoding many transmembrane polypeptides are described in the literature. According to an entirely advantageous case, the nucleic acid sequence encoding the heavy chain of the antibody is fused with the nucleic acid sequence encoding a said transmembrane polypeptide.

The expression “elements which ensure the expression of a gene in vivo” refers in particular to the elements required to ensure the expression of said gene after it has been transferred into a target cell. They are in particular promoter sequences and/or regulatory sequences which are effective in said cell, and, optionally, the sequences required to allow the expression of said polypeptide at the surface of the target cells. The promoter used may be a viral, ubiquitous or tissue-specific promoter or a synthetic promoter. By way of example, mention will be made of promoters such as the promoters of the RSV (Rous Sarcoma Virus), MPSV, SV40 (Simian Virus) or CMV (cytomegalovirus) viruses or of the vaccinia virus, and the promoters of the gene encoding muscle creatine kinase and the gene encoding actin. It is also possible to choose a promoter sequence which is specific for a given cell type or which can be activated under defined conditions. The literature provides a large amount of information relating to such promoter sequences.

The term “cytotoxic effector cells” is intended to mean macrophages, astrocytes, cytotoxic T lymphocytes (CTLs) which express CD8 molecules at their surface, and killer (NK) cells and derivatives thereof such as, for example, LAKs (Versteeg 1992 Immunology today 13: 244-247; Brittende et al. 1996, Cancer 77: 1226-1243). The term “helper T lymphocytes” is intended to denote in particular cells expressing CD4 molecules at their surface which enable, after activation, the secretion of factors for activation of the effector cells of the immune response. The polypeptides and in particular the receptors expressed at the surface of these cells, and which are involved in the activation of such cells, consist in particular of all or part of the TCR complex comprising the Vb determinants and/or CD3, all or part of the CD8, CD4, CD28, LFA-1, 4-1BB (Melero et al., 1998, Eur J Immunol 28: 1116-1121), CD47, CD2, CD1, CD9, CD45, CD30 and CD40 complexes, all or part of the receptors for cytokines (Finke et al., 1998, Gene therapy 5: 31-39), such as IL-7, IL-4, IL-2, IL-15 or GM-CSF, all or part of the receptor complex for NK cells, such as for example NKAR, Nkp46, etc. (Kawano et al., 1998 Immunology 95: 5690-5693; Pessino et al., 1998 J Exp Med 188: 953 960), Nkp44, and all or part of the macrophage receptors such as, for example the Fc receptor (Deo et al., 1997, Immunology Today 18: 127-135).

The term “therapeutic effectiveness” is generally intended to mean the inhibitory effect with respect to the superantigen-like activity as defined by the Vβ profiles of the invention and the capacity of inhibiting and/or of blocking the molecular and/or immunological interactions of the Vβs of the invention leading to their expansion or decrease as described above.

The term “supernatant” or “fraction of the supernatant” is intended to mean the supernatant, or a fraction of the latter, including the molecules, peptides and/or proteins which they comprise.

The experiments are carried out using:

1) blood mononucleated cells originating from eleven healthy donors and purified on a Ficoll gradient. The HLA DR typing for each donor was carried out beforehand.

2) (i) a pool of culture supernatants, concentrated by ultracentrifugation (100 000 g×2 hours), of human B lymphocytes originating from MS patients (MS BL) or from non-MS controls (CTBL), and (ii) culture supernatant of human choroid plexus cells originating from an MS patient (GRE) and from a non-MS patient (LES).

3) recombinant proteins.

### EXAMPLE 1

### Preparation of Extracts of Culture Supernatants of Cells Originating from Patients Suffering from MS or from Non-MS Controls

The protocols for culturing choroid plexus cells have been described in PCT patent application WO 93/20188.

1) Culturing Choroid Plexus Cells.

The choroid plexus cells are obtained from explants taken post-mortem and placed in culture, optionally after mechanical and/or enzymatic dissociation of the tissues taken. The cells which proliferate in the culture have a fibroblastic appearance and correspond to cells of leptomeningeal origin, sometimes termed “choroid plexus fibroblasts”. They can be cultured for a certain number of passages and can be frozen during intermediate passages and subsequently thawed for a new culture.

The LES (non-MS control) and GRE (MS) cells were thawed and then put back into culture in “complete” F-12 medium containing:

200 U/ml penicillin

20 mg/l streptomycin

6 mM L-glutamine

1% sodium pyruvate

1% essential amino acids

anti-leukocytic IFN antibody (polyclonal anti-alpha interferon sold by Sigma) added to 10 U/ml final.

This medium is supplemented with 30% of FCS (fetal calf serum) decomplemented at 56° C. for 30 min. The cell culturing takes place in a humidified incubator at 37° C., in the presence of 5% of CO₂. The medium is changed twice a week. If the adherent cells are at confluency at the bottom of the flask, the culture is split into two. To do this, the culture supernatant (CS) is removed and replaced with “fresh” complete F12 medium/30% FCS. The cells are detached from the flask wall using a scrapper or a trypsin/EDTA mixture. They are then carefully resuspended before being distributed into two culture flasks. The culture has been “split into two” or it has “undergone a passage”.

The CSs are harvested and frozen at −80° C. for ultracentrifugation, after elimination of the cell debris by centrifugation at 3000 rpm for 30 min. For the study, a total volume of approximately 400 ml was thawed, pooled and homogenized before ultracentrifugation, for each culture (MS/GRE and non-MS/LES).

2) Culturing Lymphoblastoid B Lines.

Production of B lymphocytes from peripheral blood and establishment of lymphoblastoid B lines immortalized with EBV (Epstein Barr Virus).

Human lymphocytes are isolated from 50 ml of heparinized blood diluted 50/50 with RPMI 1640, by centrifugation on a Ficoll gradient. They are carefully harvested from the band, as are possible cellular aggregates which may float just above the band. The cells are then washed twice in RPMI 1640 medium. After these washes, the cells are resuspended at the concentration of 2×10⁶ cells/ml in RPMI 1640 medium containing:

200 U/ml penicillin

20 mg/l streptomycin

6 mM L-glutamine

1% sodium pyruvate

1% essential amino acids

anti-leukocytic IFN antibody (polyclonal anti-alpha interferon sold by Sigma) added to 10 U/ml final.

This medium is supplemented with 20% of FCS (fetal calf serum) decomplemented at 56° C. for 30 min.

The culture flasks are incubated in incubators at 37° C. in a humidified atmosphere containing 5% of CO₂, after having added to the culture medium 1 ml of filtered EBV B95-8 productive culture supernatant (i.e. 105 EBV viral particles per 4 to 5×10⁶ total lymphocytes) in the presence of 200 μl of a solution of CSA-Sandoz (2 μg of CSA per 4 to 5×10⁶ total lymphocytes), this being until the first change of culture medium. The culture medium is changed twice a week. The flasks are maintained for three months, beyond which, if no lymphocyte proliferation has begun, the flask is discarded. When cell proliferation has started in a flask, the cells are conserved in the same flask until a significant number of proliferative colonies form clumps. The culture is “split into two” (passaged or subcultured) after mechanical dissociation of the clumps by pipetting and vigorous inverting. The cultures thus obtained correspond to B lymphocyte lines (lymphoblastoid lines). Four lines originating from definite MS patients and two lines originating from non-MS controls were obtained under these conditions.

Freezing of Cells.

The cells are washed in RPMI medium/20% FCS. The pellet taken up in FCS (approximately 1 ml per 8×10⁶ cells) is transferred to a cryotube for freezing. 200 μl of a solution of FCS DMSO prepared in advance and stored at −20° C. are added to the cell suspension (final concentration of DMSO=10%), which is then lightly vortexed. The cells are thus frozen at −80° C. in carded cotton and then stored, the following day, in nitrogen.

Thawing and Putting Cells Frozen in DMSO Back into Culture.

The cells thawed at room temperature are washed twice in 10 ml of RPMI medium/20% FCS before being put back into culture.

The four MS cultures and the two control cultures are put back into culture at the same time, with the same reagents and the same batches of media, but are cultured in separate culture laboratories (P3 and P2, respectively). The separate culturing was carried out in parallel for approximately one month, in order to harvest a volume, after pooling the separate CSs from each MS patient and from each control, of approximately 600-800 ml of each type (MSBL and CTBL). This volume was made up by systematically freezing at −80° C. the supernatants taken twice a week, after a precentrifugation at 1800 rpm for 10 min in order to sediment and recover the cells in suspension, followed by a second precentrifugation at 3000 rpm for 30 min in order to remove the cell debris.

The MS and non-MS supernatants were thawed and pooled separately and mixed (MS=MSBL pool and control=CTBL pool) before ultracentrifugation (650 ml pools). The ultracentrifugation pellets of the same pool centrifuged after distribution into separate 40 ml tubes were also pooled and mixed, before aliquoting and freezing the extract to be tested, in order to obtain an extract which was homogeneous throughout all the aliquots of the same origin.

3) Detecting Mycoplasmas Which may Contaminate the Culture.

A kit (Boehringer) based on the ELISA (Enzyme-Linked ImmunoSorbent Assay) technique was used for detecting mycoplasmas in order to guarantee the absence of mycoplasma contamination in the cultures.

4) Producing a Concentrate of Culture Supernatant by Ultracentrifugation on a Cushion of Glycerol.

The thawed supernatants cleared of cell debris beforehand are transferred into tubes for ultracentrifugation. A cushion of glycerol (PBS/30% glycerol) of a few cm is then placed in the bottom of the tube. After ultracentrifugation for two hours at 100 000×g (calculated at the middle of the tube) at 4° C. and slow deceleration (30 min), the supernatant is removed and the pellet is taken up in PBS buffer with 10% of glycerol and pooled and homogenized with the pellets from the other tubes of the same pool of origin, and the entire mixture is aliquoted in 100 μl. One aliquot is used for assaying reverse transcriptase (RT) activity and the others are stored at −80° C. The required aliquots are thawed for the assays on blood mononucleated cells.

### EXAMPLE 2

### Preparation of Blood Mononucleated Cells Originating from Eleven Healthy Donors for Which the HLA DR Typing has been Established

Donors HLA-DR typing

1- DR2-DR4

2-DR1-DR1→DR1

3- DR3-DR11 (5)

4- DR13-DR8

5- DR13(6)-DR14→DR6

6- DR2-DR2→DR2

7- DR3-DR12(5)

8- DR3-DR7

9- DR3-DR13

10- DR4-DR5

11- DR4-DR4→DR4

The blood mononucleated cells from 100 ml of blood, originating from the abovementioned eleven donors, are separated from the red blood cells on a Ficoll gradient (15 ml of Ficoll/25 ml of blood) by centrifugation for 15 min at 800 g. The cells are then washed with RPMI 1640 medium and recentrifuged under the same conditions before being aliquoted and frozen in gaseous nitrogen at the final concentration of 2×10⁷ cells/ml, in a solution of 90% of decomplemented human serum and 10% DMSO.

### EXAMPLE 3

### Culturing Blood Mononucleated Cells from Healthy Donors

After rapid thawing, the lymphocytes from healthy donors (5×10⁶ cells) are brought into contact, for 30 min at 37° C., with 100 μl of concentrated culture supernatants (i) of MSBL or of CTBL or (ii) of GRE or of LES.

After the bringing into contact, the cells are resuspended in culture medium (RPMI supplemented with 10% of decomplemented human AB serum and with gentamycin) and distributed into cupules of 24-well culture plates in a proportion of 2×10⁶ cells per ml. The viability of the cells and their immunocompetence were verified by introducing, as a control, an activation with a conventional mitogen (PHA at 5 μg/2×10⁶ cells).

### EXAMPLE 4

### Analysis of the Vβ Repertoire of the CD3 Lymphocytes by Immunodetection

The repertoire of the CD3s was analyzed using a fluorescein isothiocyanate-coupled anti-CD3 antibody and 12 biotin-coupled anti-Vβ antibodies (Immunotech-Coulter-Beckman). These twelve antibodies are listed in Table 1 below.

TABLE 1

| Ab: | | | |
|---|---|---|---|
| anti-Vβ | Reference | Sag target | Comments |
| Vβ2 | IM 2081 | TSST-1 | 10% of CD3 + PBL |
| Vβ3 | IM 2109 | SEB | |
| Vβ5-1 | IM 2082 | | 4-7% of CD3 + PBL |
| Vβ7 | IM 2288 | | 1.5-2.4% of CD3 + PBL |
| | | | IDDM diabetes superantigen |
| | | | (Conrad et al., J Immunol, |
| | | | 1997) |
| Vβ8 | IM 1191 | SEE, N | 2.6-5.1% of CD3 + PBL |
| | | rabies protein | Rabies superantigen (Lafon et |
| | | | al., Nature 1992) |
| Vβ12 | IM 2019 | SEB (12a) | 1.1-1.9% of CD3 + PBL |
| Vβ13-1 | IM 2021 | EBV | 1.8-3.3% of CD3 + PBL |
| | | | Epstein Barr superantigen |
| | | | (Sutkowski et al., J. Exp. Med. |
| | | | 1996) |
| Vβ14 | IM 2022 | | 2.2-5.6% of CD3 + PBL |
| Vβ16 | IM 2023 | | 1.1-2% of CD3 + PBL |
| Vβ17 | IM 1194 | MAM, | 3.3-7% of CD3 + PBL |
| | | SEB | |
| Vβ21-3 | IM 2025 | | 2.2-3.6% of CD3 + PBL |
| Vβ22 | IM 2026 | | 2.4-5.1% of CD3 + PBL |

After culturing for 24 hours, the content of the cupules was cultured, respectively, in the presence of MSBL, CTBL and PHA, or GRE, LES, and PHA is harvested The supernatant is aliquoted and stored at −80° C. for the subsequent detection of cytokines. The cells are washed in phosphate buffer, distributed into microtubes and incubated with a fluorescein isothiocyanate-labeled anti-CD3 antibody (0.5 μg per 5×10⁵ cells) and with each biotinylated anti-Vβ antibody (1 μg of antibody per 2×10⁶ cells) for 30 min at 4° C. simultaneously. The cells are then washed in phosphate buffer and incubated for 30 min at 4° C. with phycoerythrin-coupled streptavidin (1 μl in 50 μl of cell suspension). At the end of this incubation time, the cells are washed and resuspended in 250 μL of Cell Fix (commercial name) before being analyzed by flow cytometry. The immunofluorescence is measured using a FACSCALIBUR (commercial name) flow cytometer equipped with CellQUEST II software (commercial name) (Becton-Dickinson).

For each culture and each antibody, the percentages of cells expressing a particular Vβ among the total CD3+ cells are calculated. The size of the families is compared in the cultures activated with MSBL, CTBL, LES and GRE. A 31% increase in a Vβ family in an MSBL or GRE culture compared to the negative controls is considered to be a significant increase.

### EXAMPLE 5

### Protocol for Analyzing the Vβ Repertoire of T Lymphocytes by Molecular Biology

Extracts of culture supernatant from cells originating from patients suffering from MS or from non-MS controls are prepared as described in Example 1, and PBL cells from healthy donors are prepared as described in Example 2. The two preparations are cultured as described in Example 3 and the T cells are recovered with the PBLs, from the culture. The analysis of Vβ repertoire of the T receptor present at the surface of these cells is carried out by molecular biology as described below:

The total RNAs of the lymphoid cells recovered are extracted using conventional extraction techniques well known to those skilled in the art. A reverse transcription is carried out; complementary DNAs are thus synthesized (for example using the RT-Expand kit, Roche). Then, an amplification specific for a family of Vβ transcripts (TCRBV) is carried out by choosing primers specific for the Vb family studied (for example by choosing primers described in the literature) (Lue C et al., Am J Clin Pathol 1999 111: 683; Akatsuka Y et al., Tissue Antigens 1999 53: 122; Ryan D K et al., Mol Pathol 1997 50: 77; Lang R et al., J Immunol Methods 1997, 203: 181; Dreitz M J et al., Vet Immunol Immunopathol 1999 69: 113; Mima T et al., Biochem Biophys Res Commun 1999 263: 172; Aifantis I et al., Immunity 1998 9: 649; Deng X et al., J Biol Chem 1998 273: 23709; Lobashevsky A et al., Transplantation 1996 62: 1332; Hawke N A et al., J Immunol 1996 156: 2458; Lim S H et al., Cancer Immunol Immunother 1996 42: 69). Amplicon extension reactions are then carried out using, firstly, fluorescent TCR-BC oligonucleotides and, secondly, fluorescent TCR-BJ (13 J beta) oligonucleotides. These fluorescence-labeled amplicon products have different sizes due to the rearrangements of the genes specific for each T Vβ “x” clone and are analyzed on electrophoresis gel and the fluorescence associated with each band on the gel is read and quantified using a PhosphorImager.

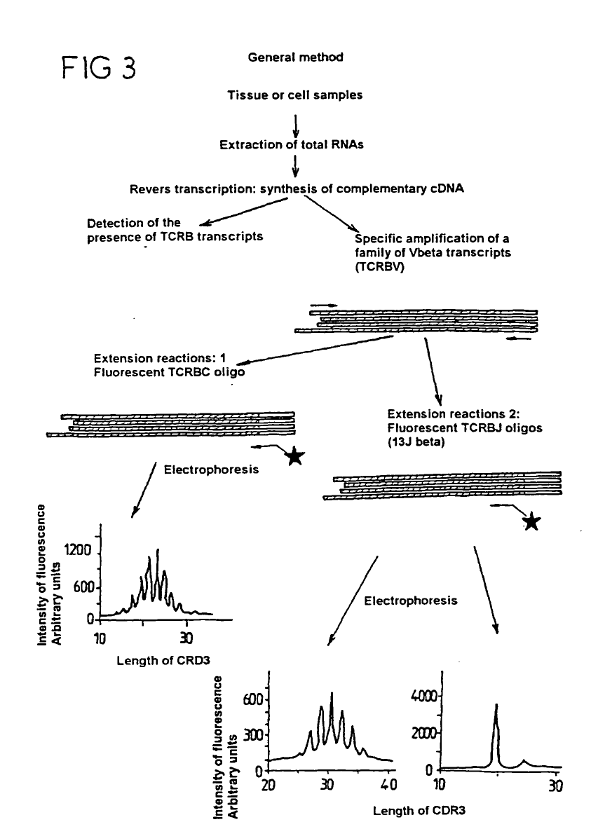

Graphs are thus obtained in which the profile reflects the amplification of a Vβ family present at the surface of T cells (figure 3). When the activation of the T cell is of the polyclonal type, several peaks are observed on the same graph, reflecting the presence of many molecules of the Vβ family at the surface of the T cells.

### EXAMPLE 6

### Detection of Cytokines Produced

The inflammatory cytokines (IL-6, TNF-α and γ-INF) were measured using Optalia (commercial name) immunocapture ELISA kits from Pharmingen-Becton-Dickinson, on 96-well titration plates according to the manufacturer's recommendations. A reference consisting of a range of dilutions of recombinant cytokine with a known concentration (in pg/ml) is included on each assay plate. The optical density is measured in a spectrophotometer at the wavelength of 405 nm for reading the chromogen ABTS (Boehringer Mannheim). The investigation of the cytokines is carried out in 50 μl of culture supernatants 24 hours after the bringing into contact for 8 donors stimulated with MSBL or CTBL, and in the culture supernatants 24 hours and 72 hours after the bringing into contact for 6 donors stimulated with LES or GRE.

The results are expressed in pg/ml of supernatant, corresponding to the production from 2×10⁶ cells.

TABLE 2

| | MSBL | CTBL |
|---|---|---|
| IL-6 (n = 6) | 714 | 220 |
| γ-INF (n = 3) | 716 | 383 |
| TNF-α (n = 6) | 83 | 54 |

### EXAMPLE 7

### Estimation of the Percentage of Cells in Apoptosis

The percentage of lymphocytes having entered into apoptosis was measured, firstly, in the PBL populations cultured for 24 hours with MSBL or CTBL, for 8 donors, and, secondly, in the populations cultured with LES and GRE, for 9 donors.

Apoptosis was estimated using a flow cytometer, taking into account the characteristics of size and of granulometry which exist between living cells and cells in apoptosis, as described for Jurkat lymphocytes (Thoulouze et al. J. Virol. 71, 7372-7380, 1997).

The percentage of apoptosis in the cultures of lymphocytes stimulated with MSBL is significantly higher than that obtained in the cultures of lymphocytes stimulated with CTBL (mean percentage apoptosis of 38.35 for MSBL against 28% for CTBL).

### EXAMPLE 8

### Detection of Viral Antigens in the Lymphocyte Cultures

Two mixtures of antibodies were used to detect the presence of viral antigens in the human lymphocytes cultured in the presence of extracts of LES and GRE choroid plexus CS, under the same conditions as for the analysis of the expansion of the TCR (T-cell receptor) Vβ families.

(i) A first pool (pool 1) of polyclonal antibodies obtained in rabbits, with 1 μl of each of the three polyclonal antibodies, and (ii) a second pool (pool 2) of monoclonal antibodies obtained after immunization of mice, with 1 μl of each of the three monoclonal antibodies, are prepared. These polyclonal and monoclonal antibodies are directed against the proteins encoded by the env, gag and pol sequences of MSRV-1, described in patent applications WO-A-98/23755 and WO-A-99/02666.

Human lymphocytes originating from healthy donors (10⁶ cells per staining) are brought into contact, for 24 hours, with ultracentrifuged supernatants of LES or GRE choroid plexus cells. The lymphocytes are then washed in RPMI medium. The cell pellet is taken up in 500 μl of fixing buffer (phosphate buffer 3% of paraformaldehyde) and incubated for 20 min at 4° C. After two washes in a permeabilization buffer (phosphate buffer containing 1% of fetal calf serum, 0.1% of sodium azide and 0.1% of saponin), the fixed cells are incubated for 30 min at 4° C. with 3 μl of each of the anti-MSRV-1 pools, in a final volume of 50 μl of permeabilization buffer. After a wash in permeabilization buffer, the cells incubated with the anti-MSRV-1 antibodies of pool 1 are incubated for 30 min at 4° C. with biotinylated antibodies directed against rabbit immunoglobulins (Byosis, dilution 1/2000), washed and then incubated for 30 min at 4° C. with fluorescein isothiocyanate-coupled streptavidin (Strep-FITC 1/50^{th}, Immunotech Coulter-Beckman). The cells incubated with the anti-MSRV-1 antibodies of pool 2 are incubated with biotin-coupled antibodies directed against mouse antibodies (Amersham, 1/500) and then, after washing, with Strep-FITC. The cells resuspended in 250 μl of Cell-fix are then analyzed by flow cytometry.

### EXAMPLE 9

### Analysis of the Vβ Repertoires of the CD3 Lymphocytes

The repertoires of eight donors are analyzed by double labeling on cells brought into contact, for 24 hours, either with MSBL or with CTBL. These same donors were tested in the presence of LES and GRE.

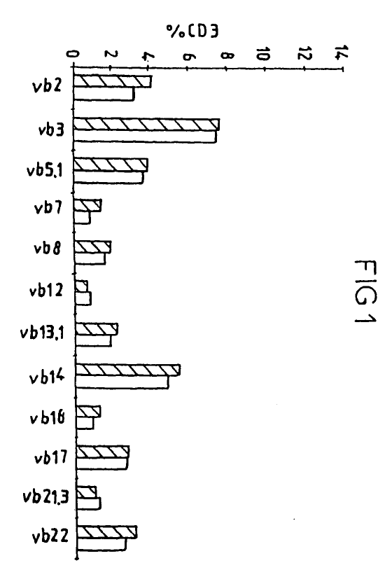

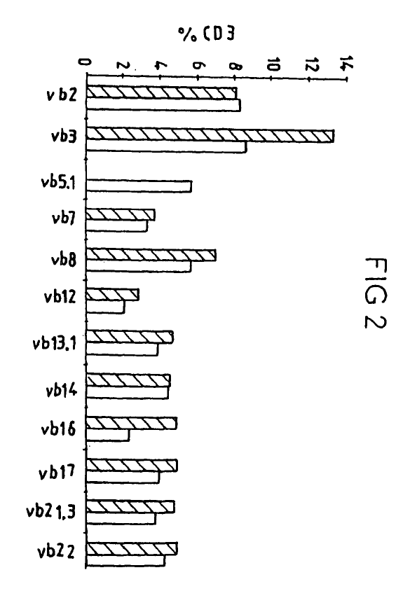

The percentage of cells expressing a particular Vβ among the CD3 lymphocytes in the cultures of lymphocytes stimulated with MSBL is compared to that obtained in the cultures stimulated with CTBL. The Vβ repertoires used by two donors (donor 1 and donor 5) in reaction to MSBL (dark histograms) and to CTBL (light histograms) are shown on figures 1 (donor 1) and 2 (donor 5). For donor 1, the percentages of Vβ used to respond to MSBL are identical to those used to respond to CTBL, with the exception of Vβ7. On the other hand, for donor 5, the percentage of Vβ3 and of Vβ16 recruited by MSBL is different to those recruited by CTBL (respectively 8.2 and 13.6 for Vβ3 and 2.1 and 4.2 for Vβ16).

A 31% increase in a Vβ family compared to the percentage obtained in the CTBL lymphocyte cultures is considered to be the mark of a significant expansion of a particular Vβ family by MSBL.

The comparative analysis using this criterion is shown in Table 3 below.

TABLE 3

| No. | HLA | Vβ2 | Vβ3 | Vβ5 | Vβ7 | Vβ8 | Vβ12 |
|---|---|---|---|---|---|---|---|
| 1 | DR 2/4 | | | | X | | |
| 4 | DR 13/8 | | | | | | X |
| 5 | DR 13/14 | | X | | | | |
| 7 | DR 3/12 | | | | | | |
| 8 | DR 3/7 | | X | | | | |
| 9 | DR 3/13 | | | | | X | |
| 10 | DR 4/5 | | X | | | | X |
| 11 | DR 4/4 | X | | | | | X |
| | % age | 12.5 | 37.5 | 0 | 13 | 13 | 37.5 |
| No. | HLA | Vβ13 | Vβ14 | Vβ16 | Vβ17 | Vβ21 | Vβ22 |
| 1 | DR 2/4 | | | | | | |
| 4 | DR 13/8 | | | X | | | |
| 5 | DR 13/14 | | | X | | | |
| 7 | DR 3/12 | | | | X | | |
| 8 | DR 3/7 | | | X | | | |
| 9 | DR 3/13 | | | X | | | |
| 10 | DR 4/5 | | X | X | | | X |
| 11 | DR 4/4 | | | X | | | |
| | % age | 0 | 12.6 | 75 | 12.5 | 0 | 12.5 |

After a contact with the MSBLs, it is observed that:

75% ( 6/8) of the donors exhibit an expansion of the Vβ16 family,

37.5% (⅜) of the donors exhibit an expansion of the Vβ12 family,

37.5% (⅜) of the donors exhibit an expansion of the Vβ3 family, and 12.5% (⅛) of the donors exhibit an expansion of one of the Vβ2, Vβ7, Vβ8, Vβ14, Vβ17 or Vβ22 families.

The results obtained with the LES and GRE preparations can be illustrated by analyzing the repertoires obtained with donors 10 and 11. GRE induces the expansion of Vβ16. The expansions obtained with GRE are of the same order and of the same nature as those obtained with MSBL.

The Vβ16 expansion is not linked to the presence of a particular DR, since it can be obtained both in a DR3 environment and in a DR4, DR5, DR8, DR13 or DR14 environment, i.e. with 6 of the 8 haplotypes expressed by the 6 donors.

MSBL generally induces, in the donors, the majority expansion of only one or two Vβ families, except in two cases: one donor exhibits both an expansion of Vβ3, Vβ12, Vβ14, Vβ16 and Vβ22, and another both Vβ12 and Vβ16. The haplotypes of these two donors are DR4/4 and DR4/5, respectively.

### EXAMPLE 10

### Analysis of the Vβ Repertoire of the T Lymphocytes by Molecular Biology

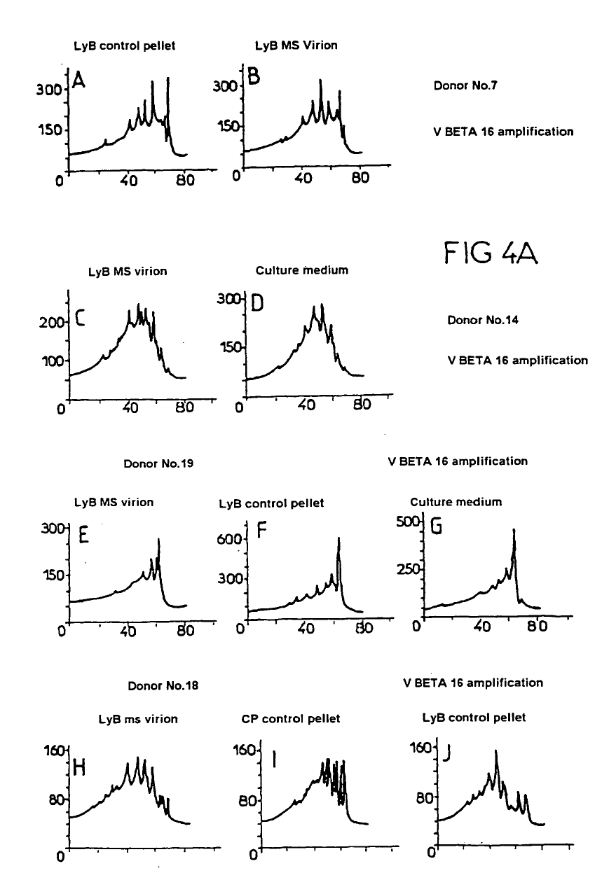

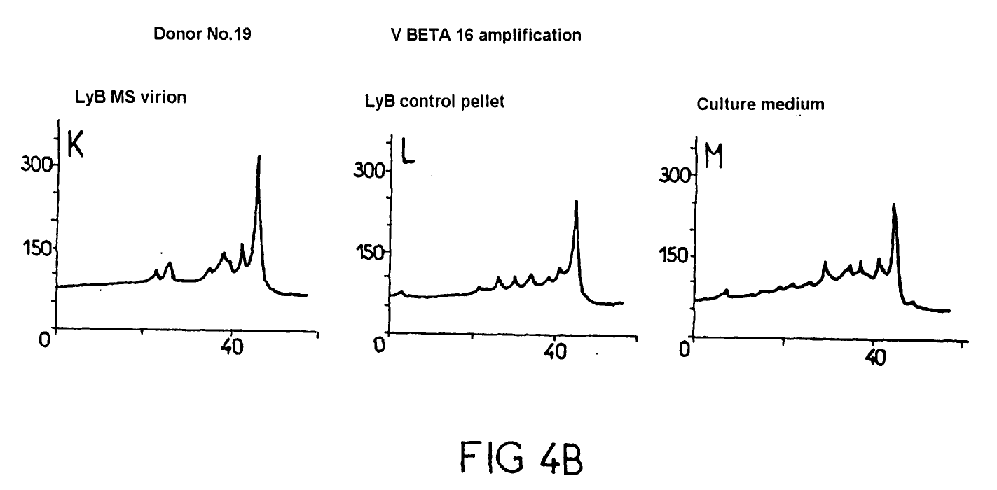

The experiment is carried out as described in Example 5. PBL cells from different healthy donors (donor 7, 14, 19 and 18) are recovered and brought into contact with centrifuged pellets from supernatants of B cells derived from MS patients or from healthy individuals. Amplifications of the Vβ16 or Vβ17 determinant are carried out (see figures 4A and 4B).

Amplification of the Vβ16 Determinant

A: Healthy donor 7 and B cells from healthy control

B: Healthy donor 7 and B cells from MS patients

C: Healthy donor 14 and B cells from MS patients

D: Healthy donor 14 and culture medium

E: Healthy donor 19 and B cells from MS patients

F: Healthy donor 19 and cells from healthy individual

G: Healthy donor 19 and culture medium

H: Healthy donor 18 and B cells from MS patient

I: Healthy donor 18 and cells from healthy individual

J: Healthy donor 18 and cells from healthy individual

Amplification of the Vβ17 Determinant

A: Healthy donor 17 and B cells from MS patient

B: Healthy donor 17 and B cells from healthy individual

C: Healthy donor 17 and culture medium

The graphs show that there is a different profile for the same Vβ16, or even Vβ17, family amplified after incubation with the B cells from MS patients or with the B cells from a healthy individual, for the same healthy PBL donor. These profiles are polyclonal. This clearly reflects the polyclonal activation of the cells from the healthy donor after incubation with pellets concentrated from supernatants of B cells from MS patients, which is different from that with pellets concentrated from supernatants of B cells from a healthy individual. This polyclonal activation reflects the presence of one or more superantigen(s) or superantigen-like material in the concentrated MS pellets. This analysis, by molecular biology, of the expansion of the T cells with a Vb16 determinant, subsequent to the incubation in the presence of concentrated MS pellets, confirms the observation made after analysis by immunodetection, directly detecting the membrane-bound Vβ determinants using specific antibodies, as described in Example 9.

### EXAMPLE 11

### Demonstration of the Expansion or of the Loss of Lymphocytes Bearing a Vβ Associated with MS

Suspensions of PBLs, peripheral blood lymphocytes, taken from healthy donors (donors 5, 7, 14, 15, 19) are brought into contact with pellets concentrated from supernatants of B cell lines (ACX, FEX, BUX) established from blood samples from MS patients as described in example 3. In parallel, these suspensions are brought into contact with pellets concentrated from supernatants of B cell lines (T8, T9) established from blood samples from healthy patients. After incubation, the Vβ repertoire of the CD3 cells is analyzed by immunodetection as described in example 4. Major proliferation of the T cells exhibiting the Vβ16 determinant is observed, as is proliferation of T cells with Vβ7, Vβ14, Vβ17 (table 4); thus, as described above, a superantigen activity is demonstrated, which has been induced by the pellets concentrated from supernatants of B lines derived from MS patients. The percentages of T-cell proliferation in vitro are estimated as described above. In parallel, it is clearly shown in this example that there may also be, with different healthy donors, a decrease in the subpopulations of T cells bearing these same Vβ16, Vβ7, Vβ14, Vβ17 for other donors. This decrease may reflect the cells being induced into a state of anergy or of apoptosis subsequent to the superantigen effect induced by the pellets concentrated from cell supernatants derived from MS patients. It should be noted that 100% of the healthy PBL suspensions tested respond either by cellular expansion or by a decrease in the number of T cells exhibiting the Vβ16 determinant, subsequent to incubation with the pellets concentrated from supernatants of B cells derived from MS patients and having superantigen activity.

The results are shown in the table below

TABLE 4

| Donor | HLA DR | Vβ2 | Vβ7 | Vβ14 | Vβ16 | Vβ17 |
|---|---|---|---|---|---|---|
| 5 | 13/14 | +7.6 | −38 | −28 | −49 | −31 |
| 7 | 3/12 | −6 | −21 | +37 | +315 | −24 |
| 14 | 4/14 | | | | +37 | +5.13 |
| 15 | 3/13 | +11 | −24 | +65 | −72 | −2 |
| 19 | 4/15 | | | | +56 | |

### EXAMPLE 12

### Stimulation of Cytokine Production

The cultures of lymphocytes stimulated with MSBL differ in that they produce significantly greater amounts of IL-6 and of γ-INF compared to those stimulated with CTBL. On the other hand, the TNF-α titers are very low and equivalent for the two types of culture. The results, expressed in pg/ml of culture corresponding to 2×10⁶ cells, are shown in Table 5.

TABLE 5

| | MSBL | CTBL |
|---|---|---|
| IL-6 (n = 6) | 714 | 220 |
| γ-INF (n = 3) | 716 | 383 |
| TNF-α (n = 6) | 83 | 54 |

### EXAMPLE 13

### Detection of Viral Antigens in the Lymphocyte Cultures

The presence of specific retroviral antigens was investigated using the two pools of antibodies directed against MSRV-1 proteins. The results of immunofluorescence on the cultures inoculated with LES or GRE ultracentrifuged CS, obtained with the two pools (pool 2=use monoclonals, pool 1=rabbit polyclonals), are shown in Table 46 for donors 10 and 11. The numbers represent the percentages of cells exhibiting fluorescence intensities ween channels 100 and 1000.

TABLE 6

| | |
|---|---|
| Donor 10 | Donor 11 |

| | LES | GRE | LES | GRE |
|---|---|---|---|---|
| Pool 1 | 0.6 | 22.6 | 0.4 | 4.76 |
| Pool 2 | 7.11 | 30.77 | 0.12 | 4.69 |

The results are given as percentages of population expressing a fluorescence intensity included between channels 100 and 1000.

The expansion, parallel to these results concerning the MSRV-1 antigens, of a restricted number of Vβ families in a majority of donors in the absence of HLA restriction indicated that the response resembles a superantigen-like response. The production of cytokines of the inflammatory type (IL-6, γ-FN) reinforces the existence of an environment which promotes lymphocyte recruitment.

The absence of TNF-α production is an argument in favor of the absence of contamination by bacterial superantigens or LPS.

### EXAMPLE 14

### Production of Monoclonal Antibodies

The production of monoclonal antibodies by ascites imposes compatibility of the H-2 system between the hybridoma and the producer mouse. 0.5 ml of Pristane (2, 6, 10, 14-tetramethylpentadecane acid) is injected into the peritoneal cavity of 6-week-old Balb/c female mice, for the production of ascites (Porter et al., 1972). One week to 10 days later 5×10⁶ to 10×10⁶ hybridomas diluted in approximately 0.5 ml of sterile 0.145 M NaCl, 10 mM Na2HPO4, 2.7 mM KCl, 1.5 mM KH2PO4 buffer, pH 7.4, are injected intraperitoneally. The ascites appear one to two weeks later. The ascites fluid present in the peritoneal cavity are then harvested with a syringe after making an incision in the peritoneum. The fluid harvested is centrifuged at 3000 g for 15 minutes at room temperature, filtered over gauze to remove the fat and then buffered by adding 1/20^{th} of its volume of 1 M tris-HCl at pH 8.0. This method makes it possible to obtain amounts of antibody which are 10 times greater than those obtained by culturing hybridomas.

The immunoglobulins present in the ascites fluid are released by salts (ammonium sulfate or sodium sulfate). The ascites fluid is precipitated with 40% ammonium sulfate. After 20 minutes in the cold, the solution is centrifuged for 15 minutes and 8000 g at 4° C. The precipitate is washed and resuspended while cold in 40% ammonium sulfate solution and then centrifuged again. The new precipitate enriched in IgG is redissolved in PBS buffer and dialyzed overnight against 25 mM Tris-HCl, 150 mM NaCl buffer, pH 7.4. In parallel, a column of agarose-Protein A (or protein G) (sold in a lyophilized form, Pierce) is washed thoroughly with the 25 mM, Tris-HCl, 150 mM NaCl buffer, pH 7.4. The solution enriched in IgG is loaded onto the column, which is then washed. The IgGs retained by the column are eluted at acid pH (200 mM glycine, pH 2.8). The fractions eluted are neutralized with a volume of 1M Tris-Base, pH 10.5. The immunoglobulin content of each fraction harvested is quantified by reading the absorbance at 280 nm (e 1%, 1 cm=14.0 Prahl and Porter, 1968). The rich fractions are pooled. The degree of purification of the pooled IgGs is analyzed by acrylamide gel electrophoresis in the presence of SDS. The purified IgGs are dialyzed overnight against the 25 mM Tris-HCl, at 150 mM NaCl buffer, pH 7.4, filtered sterilely, aliquoted and stored at −20° C. Their final concentration is determined by reading the absorbance at 280 nm or by micro-BCA assay.

However, it is within the scope of those skilled in the art to define other protocols for producing monoclonal antibodies, for example using the techniques described by Köhler and Milstein and by Galfre G. et al., previously mentioned, or techniques derived therefrom.

### EXAMPLE 15

### Measurement of the Activity of the T Cells by Cell Proliferation (Sredni et al., 1981)

The T cells are washed twice in culture medium in order to eliminate all traces of IL2 present in the initial culture medium. B lymphocytes (EBV-LCL) or monocytes/macrophages, taken as antigen-presenting cells, are irradiated at 10 000 rads and washed twice with culture medium (RPMI). 2×10⁴ T cells (2×10⁵ cell/ml) and 2×10⁴ irradiated autologous B cells (2×10⁵ cell/ml) are incubated together in the presence of a range of increasing concentration of the antigen in a final volume of 200 μl in microwells. After culturing for 48 hours at 37° C., 1 μCi of 3H-thymidine in 50 μl of RPMI medium is added to each well. The T cells, which are the only ones to divide, incorporate the tritiated thymidine into the DNA. After culturing for 18 hours, the cells of each microwell are harvested on disks of glass wool by aspiration. After osmotic lysis of the cells, the radioactivity incorporated into the DNA is absorbed onto the disks (cell Harvester 530, Inotech). Each dried disk is placed in a plastic tube which contains 2 ml of scintillant; the radioactivity b adsorbed onto each of the disks is quantified in a liquid scintillation beta counter (LKB Rackbeta 1217). The results are expressed as the arithmetic mean of cpm/culture (“counts per minute”).

### EXAMPLE 16

### Protocol for Detecting the Association Between Peptide and Histocompatibility Molecules (Approach: APCs Transformed with a Peptide Which Binds to the MHCI or MHCII Molecules)

1) Material (Example for the MHCI Molecule):

There are currently two main types of source of histocompatibility molecules: mutant cells and purified histocompatibility molecules.

The mutant cell used is the T2 human cell which is a variant of the T1 line produced by fusion of the CEM T lymphoma and of the 721.174 B lymphoma (Salter and Cresswell Embo J 1986, 5: 943-949). This cell, which lacks peptide transporters, contains heavy chains of class I molecules free of peptides which will be able to accept exogenous peptides.

Class I histocompatibility molecules purified by affinity chromatography from human B cell lines transformed with EBV may also be used. In this case, the endogenous peptides must be removed by treatment with 1.5 M urea and 12.5 mM sodium hydroxide (pH 11.7) for 1 hour at 4° C., followed by the removal thereof on a desalification column (PDLO, Pharmacia). The histocompatibility molecules are immediately put back together with the peptides to be tested in a PBS buffer with 0.05% Tween 20, 2 mM EDTA, 0.1% NP40 and 6 mM CHAPS, in the presence of 2 μg/ml B2m in order to facilitate reassociation (Gnjatic et al., Eur J Immunol 1995 25: 1638-1642). The peptides tested generally have 8 to 10 residues, sometimes 11 or 12. They were synthesized by Neosystems (Strasbourg) or by Chiron mimotopes (Victoria, Australia). They are used at concentrations ranging from 100 μM to 0.1 nM.

2) Assembly Protocol (Connan et al., Eur J Immunol 1994, 24: 777; Couillin et al. Eur J Immunol 1995, 25: 728-732).

Aliquots of 8×10⁵ cells in a volume of 64 μl, distributed in Eppendorf microfuge tubes, are mixed together with a lysis buffer containing 10 mM PBS, pH 7.5, 1% NP40 and protease inhibitors (1 mM PMSF, 100 μM iodoacetamide, 2 μg/ml aprotinin, 10 μM leupeptin, 10 μM pepstatin and 10 μg/ml trypsin inhibitor. The lysis takes place in the presence of the peptides to be tested, for 30 minutes or 1 hour at 37° C. After removal of the nonsolubilized material by centrifugation at 15 000 rpm at 4° C., 140 μl of PBS containing 0.05% of Tween 20, 3 mM of sodium azide, 1 mM PMSF and 10 mg/ml of bovine albumin are added to the supernatant. Each sample is incubated for 20 hours at 4° C. in 2 wells of a microtitration plate of the Nunc Maxisorb type, precoated with a monoclonal antibody (10 μg/ml in PBS) which recognizes the histocompatibility molecules having one or more conformation(s) corresponding to peptide presentation and similar to that (those) present at the surface of cells. The antibody-coated plate is presaturated with bovine albumin at 10 mg/ml in PBS-Tween before the sample is added. The second antibody allows the assembly of the targeted histocompatibility molecules to be detected. It is coupled either to biotin (NHS-LC biotin, Pierce) or to alkaline phosphatase (P-552, Sigma) and is incubated for one hour at 37° C. When biotin is used, a 45 minute incubation at 20-25° C. with alkaline phosphatase-coupled streptavidin (E-2636, Sigma) is carried out. The alkaline phosphatase activity is measured using, as substrate, 4-methylumbelliferyl phosphate (M-8883, Sigma) at 100 μM in 50 mM diethanolamine, pH 9.5, with 1 mM MgCl2. The reading is performed at 340/460 nm using a flow cytometer.

3) Stability of the HLA/Peptide Complexes:

The stability of the abovementioned complexes was studied since it conditions the correct presentation of the antigen and the induction of the T response. To this effect, either purified HLA or the cell lysate was used. With the purified HLA, the endogenous peptides were removed as described in 2), followed by mixing together with the peptide to be tested in Eppendorf tube at 37° C. for variable periods of time from a few minutes to several days. The subsequent phase of incubation on a 96-well plate (as described in 2) with the anti-HLA antibody takes place for one hour at 37° C. The revelation is carried out conventionally. With the cell lysate, all the incubations are also carried out at 37° C., after adding all the protease inhibitors.

### EXAMPLE 17

### The MSRV Envelope Protein Reproduces the Dominant Stimulation of the Population of T Lymphocytes Bearing Vβ16 Chains, Observed Beforehand with the Infection with the Fraction Containing the Viral Particles

The expansion or the depletion (loss) of the subpopulations of T lymphocytes was analyzed by FACS on blood mononucleated cells from healthy donors overall according to the protocols described in Examples 2, 3, 4, 9 and 11.

Table 7 shows the results obtained with a new batch MS virion produced in MS choroid plexus cultures, which was tested on a new series of various healthy donors. It shows the reproducibility of the majority Vβ16 stimulation in most of the healthy donors (Don 12, 15, 16, 19 and 20). The weak reactivity of two donors (Don 14 and 18) corresponds to a low percentage of “non-responder” donors regularly observed in the successive series. The parallel decrease in all the Vβs in donor 13 is reminiscent of a problem of viability of its cells stored in nitrogen and thawed for culturing. Consequently, no further use was subsequently made of the latter donor.

TABLE 7

| | Vβ2 | Vβ14 | Vβ16 | Vβ17 |
|---|---|---|---|---|
| Don 12 | −6 | −8 | 50 | −8 |
| Don 13 | −39 | −27 | −20 | −52 |
| Don 14 | −19 | −23 | 12 | −4 |
| Don 15 | −30 | 0 | 75 | −16 |
| Don 16 | −50 | −66 | 88 | −51 |
| Don 18 | −25 | −31 | 5 | −17 |
| Don 19 | −9 | −14 | 43 | −50 |
| Don 20 | 0 | 17 | 67 | 0 |

The modification made to the protocols described in the examples named above consisted in replacing the addition of the fraction containing the virions produced by MS cell cultures, with recombinant proteins produced from the MSRV retroviral sequences associated with these same sources of virions or with the solubilization buffer alone.

This analysis has already made it possible to demonstrate the immunological properties of the envelope protein of the MSRV retrovirus (MSRV env) reproducing the major stimulation of the Vβ16-positive T-lymphocyte subpopulation and a minor stimulation of the Vβ17-positive T-lymphocyte subpopulation.

To do this, a bacterial expression plasmid was obtained with the DNA insert encoding the reading frame of an MSRV env protein, according to the molecular biology techniques known to those skilled in the art.

The sequence of the insert is referenced in SEQ ID No. 1.

The amino acid sequence of the recombinant protein expressed from this plasmid is referenced in SEQ ID No. 2.

The expression plasmid made it possible to produce the MSRV env protein fused with a “polyhistidine” ending (HIS-Tag) which made it possible to purify it, on an affinity column, from the extract of recombinant E. coli bacteria in which the protein was expressed.

The recombinant env protein was purified and analyzed by SDS PAGE electrophoresis, in the presence of molecular weight markers. The fractions of elution specific for the recombinant protein produced in the insoluble fraction retained, via the HIS-Tag, on an affinity column were recovered. It was confirmed that this corresponded to the protein fused to the HIS-Tag by Western blot, with an antibody specific for the HIS-Tag, which labeled the 60 KD protein found alone in the eluted fraction, after visualization on an SDS PAGE electrophoresis gel. The richest fraction (approximately 0.5 mg/ml) was used for adding to the cells from healthy donors in the immunological assay which is the subject of this example.

The blood mononucleated cells from three donors (12, 15 and 16) were incubated in parallel for 3 days in the presence of 10 ng/ml and of 100 ng/ml of the recombinant MSRV env protein, and of an equivalent dilution of the solubilization buffer alone.

After incubation for these three days, the analysis of the Vβ repertoire showed, according to the analytical criteria already described in the examples above, that donors 12 and 16 responded significantly, by an expansion of Vβ16 and Vβ17, to the presence of the MSRV env protein. Donor 16 also showed a Vβ2 expansion. No directed Vβ response was found in donor 15.

The results are summarized in Table 8.

TABLE 8

| | Vβ2 | Vβ7 | Vβ14 | Vβ16 | Vβ17 |
|---|---|---|---|---|---|
| Don 12 | | | | | |
| 10 ng/ml | 13 | −7 | 7 | 159 | 151 |
| 100 ng/ml | 9 | −9 | 8 | 212 | 156 |
| Don 15 | | | | | |
| 10 ng/ml | 10 | 4 | 32 | −30 | 24 |
| 100 ng/ml | 7 | 2 | 1 | −4 | 6 |
| Don 16 | | | | | |
| 10 ng/ml | 80 | −60 | ND | 52 | 156 |

This indicates that the MSRV env protein may be responsible for the dominant stimulation of the Vβ16 subpopulations, observed with the total viral fraction containing the MSRV-1 particles which was used in the examples above, but also for the costimulations or alternative stimulations of some other subpopulations (ex. Vβ17 and Vβ2), observed with a lesser frequency over all the healthy donors tested to date.

These results also indicate that the MSRV-1 sequences which encode the env protein can be taken from the nucleotide sequences described above and that the MSRV-1 env protein variants can be taken from the sequences described previously, given that the retroviral sequence variability does not exclude the stability of a particular biological property over a majority of variants.

### EXAMPLE 18

### Abolition of Superantigen Stimulation of the T-Lymphocyte Subpopulations, by Addition of Antiviral Medicinal Products

The expansion or the depletion of the T-lymphocyte subpopulations was analyzed by FACS on blood mononucleated cells from healthy donors, according to the description given in Examples 2, 3, 4, 9 and 11.

The modification made to the protocol described in the examples named above consists in inoculating, in parallel, in wells containing the blood mononucleated cells from the same healthy donor: 1) a fraction containing the virions produced by MS cell cultures, 2) the same viral fraction in the presence of 12.5 μM AZT (Sigma), 3) the same viral fraction in the presence of 20 μM DDI (Sigma). These cultures were prepared on the cells from a donor whose blood mononucleated cells had been frozen in aliquots and for whom one or more aliquots had shown a superantigen-like effect in the presence of virions originating from MS cultures (cf. Table 7).

Moreover, toxicity tests with AZT (azidodeoxythymidine) alone and DDI (dideoxyinosine) alone were carried out on the same cells and showed no significant cell mortality.

The study with antiretroviral medicinal products has already made it possible to demonstrate the inhibitory action of this type of medicinal product on the induction of a superantigen-like stimulation by a concentrated fraction of culture supernatants of cells originating from patients suffering from MS and containing retroviral particles associated with MSRV-1 RNA. The corresponding results are shown in Table 10 below. The row entitled “medium” corresponds to the medium containing the MS virion without antiretroviral agents and the rows entitled “DDI” and “AZT” refer to the tests carried out in the presence of these inhibitors. The FACS analysis of the T subpopulations in the various culture wells, carried out according to the examples above, shows:

a significant Vβ16 expansion of the lymphocytes incubated in the presence of the “MS” virion alone, which was not found in the presence of the “virion control” fraction originating from “non-MS” control cultures.

an absence or an inhibition of Vβ16 expansion of the lymphocytes incubated in the presence of the “MS” virion and of AZT.

an absence of Vβ16 expansion of the lymphocytes incubated in the presence of the “MS” virion and of DDI.

TABLE 10

| | Vβ2 | Vβ14 | Vβ16 | Vβ17 |
|---|---|---|---|---|
| Don 12 | | | | |
| AZT | 13 | −6 | 7 | 0 |
| DDI | −4 | −21 | −15 | −11 |
| Medium | −6 | −8 | 50 | −8 |
| Don 20 | | | | |
| AZT | −3 | −15 | −3 | 16 |
| DDI | −5 | −35 | −33 | −19 |
| Medium | 0 | 17 | 67 | 0 |

In view of these results, now it appears that any therapeutic method, molecule or medicinal product which has an inhibitory, blocking or modulating effect on retroviral expression, in particular infectious and/or endogenous retroviral expression, may be used to inhibit the immunopathological effect induced by the pathogenic agent(s) associated with the production of these retroviral particles. The general characteristics of the immunopathological effect are described in the present invention.

In view of these results and of those of Examples 8 and 17, it now appears that these therapeutic methods or agents may be chosen from the compounds which are active on the expression of the MSRV-1 retrovirus.

### BIBLIOGRAPHICAL REFERENCES REGARDING VβS

Brenden N, et al., Differential MHC expression requirements for positive selection of separate TCR Vb families,. Immunogenetics. 1999 January; 49(1): 1-6.

Hodges E, et al., T cell receptor (TCR) Vbeta gene usage in bronchoalveolar lavage and peripheral blood T cells from asthmatic and normal subjects,. Clin Exp Immunol. 1998 June; 112(3): 363-74.

Allen T M, et al., The T-cell receptor beta chain-encoding gene repertoire of a New World primate species, the cotton-top tamarin, Immunogenetics. 1996; 45(2): 151-60.

Yassai M, et al., Bacterial toxin superantigens stimulate all members of susceptible VB gene families, Ann N Y Acad Sci. 1995 Jul. 7; 756: 110-2.

Donahue J P, et al., Genetic analysis of low V beta 3 expression in humans,. J Exp Med. 1994 May 1; 179(5): 1701-6.

Isono T, et al., Sequence and diversity of variable gene segments coding for rabbit T-cell receptor beta chains,. Immunogenetics. 1994; 39(4): 243-8.

Levinson G, et al., Sequence and diversity of rhesus monkey T-cell receptor beta chain genes, Immunogenetics. 1992; 35(2): 75-88.

Buitkamp J, et al., Vb6 T-cell receptor elements in artiodactyls: conservation and germline polymorphisms, Mamm Genome. 1993 September; 4(9): 504-10.

Johnston S L, et al., Diversity of alpha and beta subunits of T-cell receptors specific for the H4 minor histocompatibility antigen, Immunogenetics. 1997; 46(1): 17-28.

Huck S, et al., Variable region genes in the human T-cell rearranging gamma (TRG) locus: V-J junction and homology with the mouse genes, EMBO J. 1988 March; 7(3): 719-26.

Sherman L A, et al., Comparison of the H-2Kb-specific cytolytic T lymphocyte receptor repertoire in Igh recombinant strains, J Immunol. 1985 June; 134(6): 3569-73.

1. A method for detecting multiple sclerosis (MS) or a condition associated with multiple sclerosis, in a biological sample, wherein a superantigen activity is detected, said superantigen activity being induced by the expression product of the env gene of MSRV-1 comprising the sequence referenced in SEQ ID NO: 1, or a variant thereof having at least 90% sequence identity with SEQ ID NO: 1, or a fragment of SEQ ID NO: 1, said method comprising:
detecting an amount of lymphocytes bearing a Vβ16 and/or Vβ17 determinant in said biological sample; and
calculating an amount of expansion or loss of lymphocytes bearing a Vβ16 and/or Vβ17 determinant based on said detected amount,
wherein a majority expansion of lymphocytes bearing a Vβ16 and/or Vβ17 determinant or a majority loss of lymphocytes bearing a Vβ16 and/or Vβ17 determinant indicates the superantigen activity.

2. The detection method as claimed in claim 1, wherein a majority expansion of lymphocytes bearing a Vβ16 determinant is demonstrated.

3. The detection method as claimed in claim 1, wherein a majority loss of lymphocytes bearing a Vβ16 determinant is demonstrated.

4. The method as claimed in claim 2, wherein a majority expansion of lymphocytes bearing a Vβ16 determinant and a co-expansion of lymphocytes bearing Vβs chosen from at least any one of Vβ2, Vβ3, Vβ7, Vβ8, Vβ12, Vβ14, Vβ17 and Vβ22 are demonstrated.

5. The method as claimed in claim 3, wherein a majority loss of lymphocytes bearing a Vβ16 determinant and a co-decrease of lymphocytes bearing Vβs chosen from at least any one of Vβ2, Vβ3, Vβ7, Vβ8, Vβ12, Vβ14, Vβ17 and Vβ22 are demonstrated.

6. The method as claimed in claim 1, wherein the biological sample originates from a patient suffering from multiple sclerosis.

7. The method as claimed in claim 1, wherein:
(i) a culture supernatant of blood mononucleated cells or of choroid plexus cells or of leptomeningeal cells, said cells originating from patients suffering from multiple sclerosis or of an established cell line, is sampled, and
(ii) said culture supernatant, or a part of the culture supernatant is brought into contact with a series of cultures of blood mononucleated cells originating from healthy donors, and
(iii) said expansion and, optionally, a co-expansion, or said loss and, optionally, co-decrease, of the blood mononucleated cells of step (ii) are detected.

8. The method as claimed in claim 7, wherein the blood mononucleated cells originating from patients suffering from multiple sclerosis are chosen from monocytes and B lymphocytes and the blood mononucleated cells originating from healthy donors are chosen from T lymphocytes.

9. The method as claimed in claim 1, wherein:
(i) blood mononucleated cells are sampled, said cells originating from patients suffering from multiple sclerosis and from healthy individuals,
(ii) said blood mononucleated cells originating from patients or from healthy individuals are brought into contact with culture supernatants, or a fraction of culture supernatant, of cells chosen from blood mononucleated cells, choroid plexus cells and leptomeningeal cells, and cells derived from established cell lines, and
(iii) said expansion and, optionally, co-expansion, or said loss and, optionally, co-decrease, using the blood mononucleated cells of step (i) are detected.

10. The method as claimed in claim 7, wherein said expansion and, optionally, co-expansion is demonstrated using ligands, each ligand being specific for a determinant chosen from Vβ16, Vβ2, Vβ3, Vβ7, Vβ8, Vβ12, Vβ14, Vβ17 and Vβ22, and in that said loss and, optionally, co-decrease is demonstrated using ligands, each ligand being specific for a determinant chosen from Vβ16, Vβ2, Vβ3, Vβ7, Vβ8, Vβ12, Vβ14, Vβ17 and Vβ22.

11. The method as claimed in claim 10, wherein the ligand is an antibody or antibody fragment.

12. The method as claimed in claim 7, wherein, in order to demonstrate said expansion and, optionally, co-expansion or said loss and, optionally, co-decrease, the following is carried out:
(i) extraction of the total RNAs from the blood mononucleated cells which have been placed together with MS culture supernatant or a fraction of MS culture supernatant and together with control culture supernatant or a fraction of control culture supernatant,
(ii) reverse transcription of said RNAs,
(iii) amplification specific for each Vβ family using a given pair of primers,
(iv) labeling of the amplification products obtained, with any suitable label,
(v) electrophoresis of said amplification products and analysis of the electrophoretic profiles obtained, using a suitable detector.

13. The method as claimed in claim 12, wherein the blood mononucleated cells originating from patients suffering from MS are chosen from lymphocytes.

14. The method as claimed in claim 1, wherein the superantigen activity is induced by the envelope protein of MSRV-1 referenced in SEQ ID NO: 2 or by a fragment of said protein.

15. The method as claimed in claim 1, wherein the superantigen activity is induced by the env gene of MSRV-1 referenced in SEQ ID NO: 1 or a fragment of said gene.

16. A method for detecting multiple sclerosis (MS) or a condition associated with multiple sclerosis, wherein a superantigen activity is detected, said superantigen activity being induced by the expression product of the env gene of MSRV-1 comprising the sequence referenced in SEQ ID NO: 1, or a variant thereof having at least 90% sequence identity with SEQ ID NO: 1, or a fragment of SEQ ID NO: 1, said method comprising:
detecting an amount of lymphocytes bearing a Vβ7 determinant in said biological sample; and
calculating an amount of expansion or loss of lymphocytes bearing a Vβ7 determinant based on said detected amount,
wherein a majority expansion of lymphocytes bearing a Vβ7 determinant or a majority loss of lymphocytes bearing a Vβ7 determinant indicates the superantigen activity.

17. The method as claimed in claim 16, wherein:
(i) a culture supernatant of blood mononucleated cells or of choroid plexus cells or of leptomeningeal cells, said cells originating from patients suffering from multiple sclerosis or of an established cell line, is sampled, and
(ii) said culture supernatant, or a part of the culture supernatant is brought into contact with a series of cultures of blood mononucleated cells originating from healthy donors, and
(iii) said expansion and, optionally, a co-expansion, or said loss and, optionally, co-decrease, of the blood mononucleated cells of step (ii) are detected.

18. The method as claimed in claim 17, wherein the blood mononucleated cells originating from patients suffering from MS are chosen from B lymphocytes and monocytes and the blood mononucleated cells originating from healthy donors are chosen from T lymphocytes.

19. The method as claimed in claim 16, wherein:
(i) blood mononucleated cells are sampled, said cells originating from patients suffering from multiple sclerosis and from healthy individuals,
(ii) said blood mononucleated cells originating from patients or from healthy individuals are brought into contact with culture supernatants, or a fraction of culture supernatant, of cells chosen from blood mononucleated cells, choroid plexus cells and leptomeningeal cells, and cells derived from established cell lines, and
(iii) said expansion and, optionally, co-expansion, or said loss and, optionally, co-decrease, using the blood mononucleated cells of step (i) are detected.

20. The method as claimed in claim 16, wherein:
(i) a culture supernatant of blood mononucleated cells or of choroid plexus cells or of leptomeningeal cells, said cells originating from patients suffering from multiple sclerosis or of an established cell line, is sampled, and
(ii) said culture supernatant, or a part of the culture supernatant is brought into contact with a series of cultures of blood mononucleated cells originating from healthy donors, and
(iii) said expansion and, optionally, a co-expansion, or said loss and, optionally, co-decrease, of the blood mononucleated cells of step (ii) are detected using a ligand or amplification combined with electrophoresis.

21. The method as claimed in claim 1, wherein:
(i) a polypeptide as identified by SEQ ID NO: 2, or a fragment of said polypeptide, is produced or synthesized, said fragment being at least six amino acids in length,
(ii) said polypeptide is brought into contact with a series of cultures of blood mononucleated cells originating from healthy donors, and
(iii) said expansion and, optionally, a co-expansion, or said loss and, optionally, co-decrease, of the blood mononucleated cells of step (ii) are detected.

22. The method as claimed in claim 1, wherein:
(i) blood mononucleated cells are sampled, said cells originating from patients suffering from multiple sclerosis and from healthy individuals,
(ii) said blood mononucleated cells originating from patients or from healthy individuals are brought into contact with a polypeptide or a recombinant protein, as identified in SEQ ID NO: 2, or a fragment of said polypeptide, said fragment being at least six amino acids in length, and
(iii) said expansion and, optionally, co-expansion, or said loss and, optionally, co-decrease, using the blood mononucleated cells of step (i) are detected.

23. The method as claimed in claim 22, wherein a polypeptide comprising at least one or more fragment(s) of the env protein of MSRV-1 identified by SEQ ID NO: 2, said fragment being at least eight amino acids in length, is used.

24. The method as claimed in claim 21, wherein said polypeptide is encoded by a nucleic acid comprising at least one or more fragment(s) of the RNA or of the DNA of the env gene of MSRV-1, identified by SEQ ID NO: 1, said fragment being at least 18 nucleotides in length, or a vector comprising said nucleic acid.

25. The method as claimed in claim 1, wherein to calculate said amount of expansion or loss, the detected amount is compared to an amount of lymphocytes bearing a Vβ16 and/or Vβ17 in a biological sample of at least one healthy individual.

26. The method as claimed in claim 4, wherein a co-expansion of lymphocytes bearing at least one of Vβ3 and Vβ12 is demonstrated.

27. The method as claimed in claim 5, wherein a co-decrease of lymphocytes bearing at least one of Vβ7, Vβ14 and Vβ17 is demonstrated.

28. The method as claimed in claim 27, wherein a co-decrease of lymphocytes bearing at least one of Vβ7 and Vβ17 is demonstrated.

29. The method as claimed in claim 7, wherein said established cell line is the PLI-2 cell line deposited at the ECACC on Jul. 22, 1992, under the number 92072201, or the LM7PC cell line deposited at the ECACC on Jan. 8, 1993, under the number 93010817, in accordance with the provisions of the Budapest Treaty.

30. The method as claimed in claim 7, wherein said series of cultures comprise at least three cultures.

31. The method as claimed in claim 9, wherein said established cell line is the PLI-2 cell line deposited at the ECACC on Jul. 22, 1992, under the number 92072201, or the LM7PC cell line deposited at the ECACC on Jan. 8, 1993, under the number 93010817, in accordance with the provisions of the Budapest Treaty.

32. The method as claimed in claim 9, wherein said expansion and, optionally, co-expansion is demonstrated using ligands, each ligand being specific for a determinant chosen from Vβ16, Vβ2, Vβ3, Vβ7, Vβ8, Vβ12, Vβ14, Vβ17 and Vβ22, and in that said loss and, optionally, co-decrease is demonstrated using ligands, each ligand being specific for a determinant chosen from Vβ16, Vβ2, Vβ3, Vβ7, Vβ8, Vβ12, Vβ14, Vβ17 and Vβ22.

33. The method as claimed in claim 32, wherein said expansion and, optionally, co-expansion is demonstrated using ligands, each ligand being specific for a determinant chosen from Vβ16, Vβ3 and Vβ12.

34. The method as claimed in claim 32, wherein said loss and, optionally, co-decrease is demonstrated using ligands, each ligand being specific for a determinant chosen from Vβ16, Vβ7, Vβ14 and Vβ17.

35. The method as claimed in claim 10, wherein said expansion and, optionally, co-expansion is demonstrated using ligands, each ligand being specific for a determinant chosen from Vβ16, Vβ3 and Vβ12.

36. The method as claimed in claim 10, wherein said loss and, optionally, co-decrease is demonstrated using ligands, each ligand being specific for a determinant chosen from Vβ16, Vβ7, Vβ14 and Vβ17.

37. The method as claimed in claim 11, wherein the antibody is a monoclonal antibody.

38. The method as claimed in claim 32, wherein the ligand is an antibody or antibody fragment.

39. The method as claimed in claim 38, wherein said antibody is a monoclonal antibody.

40. The method as claimed in claim 9, wherein, in order to demonstrate said expansion and, optionally, co-expansion or said loss and, optionally, co-decrease, the following is carried out:
(i) extraction of the total RNAs from the blood mononucleated cells which have been placed together with MS culture supernatant or a fraction of MS culture supernatant and together with control culture supernatant or a fraction of control culture supernatant,
(ii) reverse transcription of said RNAs,
(iii) amplification specific for each Vβ family using a given pair of primers,
(iv) labeling of the amplification products obtained, with any suitable label,
(v) electrophoresis of said amplification products and analysis of the electrophoretic profiles obtained, using a suitable detector.

41. The method as claimed in claim 40, wherein the blood mononucleated cells originating from patients suffering MS are chosen from lymphocytes.

42. The method as claimed in claim 16, wherein to calculate said amount of expansion or loss, the detected amount is compared to an amount of lymphocytes bearing Vβ7 in a biological sample of at least one healthy individual.

43. The method as claimed in claim 17, wherein said established cell line is the PLI-2 cell line deposited at the ECACC on Jul. 22, 1992, under the number 92072201, or the LM7PC cell line deposited at the ECACC on Jan. 8, 1993, under the number 93010817, in accordance with the provisions of the Budapest Treaty.

44. The method as claimed in claim 19, wherein said established cell line is the PLI-2 cell line deposited at the ECACC on Jul. 22, 1992, under the number 92072201, or the LM7PC cell line deposited at the ECACC on Jan. 8, 1993, under the number 93010817, in accordance with the provisions of the Budapest Treaty.

45. The method as claimed in claim 20, wherein the ligand is an antibody or an antibody fragment.

46. The method as claimed in claim 45, wherein said antibody is a monoclonal antibody.

47. The method as claimed in claim 20, wherein, in order to demonstrate said expansion and, optionally, co-expansion or said loss and, optionally, co-decrease, the following is carried out:
(i) extraction of the total RNAs from the blood mononucleated cells which have been placed together with MS culture supernatant or a fraction of MS culture supernatant and together with control culture supernatant or a fraction of control culture supernatant,
(ii) reverse transcription of said RNAs,
(iii) amplification specific for each Vβ family using a given pair of primers,
(iv) labeling of the amplification products obtained, with any suitable label,
(v) electrophoresis of said amplification products and analysis of the electrophoretic profiles obtained, using a suitable detector.

48. The method as claimed in claim 21, wherein said polypeptide is a recombinant protein.

49. The method as claimed in claim 21, wherein said series of cultures comprises at least three cultures.

50. A method for detecting multiple sclerosis or a condition associated with multiple sclerosis, in a biological sample from a patient having or suspected of having multiple sclerosis or having a risk for developing multiple sclerosis, said method comprising:
detecting an amount of lymphocytes bearing a Vβ16 and/or Vβ17 determinant in said biological sample; and
calculating an amount of expansion or loss of lymphocytes bearing a Vβ16 and/or Vβ17 determinant based on said detected amount,
wherein a majority expansion of lymphocytes bearing a Vβ16 and/or Vβ17 determinant or a majority loss of lymphocytes bearing a Vβ16 and/or Vβ17 determinant is indicative of multiple sclerosis or a condition associated with multiple sclerosis.

51. A method for detecting multiple sclerosis or a condition associated with multiple sclerosis, in a biological sample from a patient having or suspected of having multiple sclerosis or having a risk for developing multiple sclerosis, said method comprising:
detecting an amount of lymphocytes bearing a Vβ7 determinant in said biological sample; and
calculating an amount of expansion or loss of lymphocytes bearing a Vβ7 determinant based on said detected amount,
wherein a majority expansion of lymphocytes bearing a Vβ7 determinant or a majority loss of lymphocytes bearing a Vβ7 determinant is indicative of multiple sclerosis or a condition associated with multiple sclerosis.
